(19) United States
(12) Patent Application Publication
(10) Pub. No.: US 2023/0295116 A1
(43) Pub. Date: Sep. 21, 2023

(54) PROTEASOME ENHANCERS AND USES THEREOF

(71) Applicant: Board of Trustees of Michigan State University, East Lansing, MI (US)

(72) Inventors: Jetze J. Tepe, East Lansing, MI (US); Allison Vanecek, East Lansing, MI (US)

(21) Appl. No.: 18/041,179

(22) PCT Filed: Aug. 10, 2021
(86) PCT No.: PCT/US2021/045440

§ 371 Date: Feb. 9, 2023

Related U.S. Application Data
(60) Provisional application No. 63/064,262, filed on Aug. 11, 2020.

(51) Int. Cl. C07D 401/12; C07D 235/24

(52) U.S. Cl. CPC C07D 401/12; C07D 235/24

(57) ABSTRACT

Described herein are compounds astemizole derivatives, methods for making such compounds, and the use of such compounds in the treatment of cancer, an inflammatory disease or condition or neurodegenerative diseases, such as Parkinson’s disease, Alzheimer’s disease, Huntington’s disease, and ALS.

### CROSS-REFERENCE TO RELATED APPLICATIONS

This application claims the benefit of U.S. Provisional Appl. Ser. No. 63/064,262, filed Aug. 11, 2020, the entirety of which is incorporated by reference as if fully set forth herein.

### STATEMENT OF GOVERNMENT SUPPORT

This invention was made with government support under NS111347, AG061306 and GM092715 awarded by the National Institutes of Health. The government has certain rights in the invention.

### BACKGROUND

The regulation of protein synthesis, degradation, folding, trafficking and aggregation within a cell are collectively known as proteostasis. Proteostasis is maintained by a wide array of cellular machinery that work to ensure that proteins are present in the proper location, amounts and form to perform their respective functions. When one of the pathways involved with proteostasis becomes dysregulated there can be disastrous effects on the cell and even on neighboring cells. One increasingly prevalent example of this is seen in neurodegenerative diseases, such as Parkinson’s disease (PD), Alzheimer’s disease (AD), Huntington’s disease (HD) and amyotrophic lateral sclerosis (ALS). In these neurodegenerative diseases, accumulation of specific aggregation-prone proteins (hereafter referred to as intrinsically disordered proteins (IDPs)) leads to toxic signaling and disruption of proteostasis caused by their uncontrolled aggregation and oligomerization (hereafter, aggregation and oligomerization are used interchangeably). For example, the IDP α-synuclein (α-syn) and its oligomers are associated with the pathogenesis of PD. IDPs are named for their lack of tertiary structure allowing them to adopt numerous conformations and interact with multiple binding partners. IDPs are generally short-lived signaling proteins or transcription factors that are highly bound to other cellular components keeping free cytosolic levels low. Additionally, unbound IDPs are readily degraded by the 20S proteasome, the default protease responsible for IDP digestion. The accumulation of IDPs seen in neurodegenerative diseases can begin as a result of one of several disruptions (e.g. mutations, changes in expression, oxidative stress, aging, proteasome impairment, etc.) to their normal regulation. While α-syn may not be the sole cause of PD, there is strong evidence supporting its key role in the disease, including familial forms of PD resulting from mutations in the SNCA gene. Elevated monomeric α-syn levels are also known to cause apoptosis-inducing aggregation in neurons. Additionally, oligomeric forms of α-syn and other IDPs have recently been shown to directly inhibit the proteasome, further disrupting its ability to regulate IDPs concentrations. These data collectively suggest that the accumulation of α-syn and formation of oligomeric species of the IDP play a critical role in the progression of PD. Due to a lack of defined binding pockets, IDPs such as α-syn, and their aggregation are difficult to target through traditional small molecule drug design. There are currently no effective treatments to hinder the progression of neurodegenerative diseases that are associated with IDP accumulation.

### BRIEF DESCRIPTION OF THE FIGURES

The drawings illustrate generally, by way of example, but not by way of limitation, various embodiments discussed herein.

FIG. 1A is the structure of astemizole, which is referred to herein as “compound 1,” and acyl astemizole, which is referred to herein as “compound 2.”

FIG. 1B is a plot of percent fold activation over vehicle as a function of log[M] astemizole and acyl astemizole and includes two tables describing each compound’s increase activity by 200% (AC₂₀₀) and max fold increase.

FIG. 2A is a representative silver stain illustrating induced degradation of α-synuclein by the 20S proteasome in the presence of 1, 3, 5, and 10 µM astemizole (1).

FIG. 2B is a bar graph showing percent α-synuclein remaining in the presence of various concentrations of astemizole and the 20S proteasome (n=3).

FIG. 2C is a table showing the quantification of α-synuclein remaining after treatment with the 20S proteasome and various concentrations of astemizole. Error bars denote standard deviation. Ordinary one-way ANOVA statistical analysis was used to determine statistical significance (ns=not significant, *p<0.05, **p<0.01, ***p<0.001, ****p<0.0001).

FIG. 3A is a representative silver stain illustrating induced degradation of α-synuclein by the 20S proteasome in the presence of 1, 3, 5, and 10 µM acyl astemizole (2).

FIG. 3B is a bar graph showing percent α-synuclein remaining in the presence of various concentrations of acyl astemizole and the 20S proteasome (n=3).

FIG. 3C is a table showing the quantification of α-synuclein remaining after treatment with the 20S proteasome and various concentrations of acyl astemizole. Error bars denote standard deviation. Ordinary one-way ANOVA statistical analysis was used to determine statistical significance (ns=not significant, *p<0.05, **p<0.01, ***p<0.001, ****p<0.0001).

FIG. 4A is tables and accompanying plots of % luminescence as a function of astemizole concentration.

FIG. 4B is tables and accompanying plots of % luminescence as a function of acyl astemizole concentration.

FIG. 4C is tables and accompanying plots of % luminescence as a function of AV-1-10 concentration.

FIG. 5 is the chemical structure of the compound AV-1-10 mentioned in FIG. 4C.

### SUMMARY

The disclosure relates to small molecules that enhance proteasome function and restore the activity of impaired proteasomes. Small molecule proteasome enhancers prevent the toxic accumulation of aggregation-prone proteins and prevent neuronal cell death caused by aggregation-prone proteins. The disclosure therefore relates to the use of small molecules as therapeutic agents to treat neurodegenerative diseases. Neurodegenerative diseases include, but are not limited to Alzheimer’s disease (AD) and other dementias, Parkinson’s disease (PD) and PD-related disorders, Prion disease, Motor neuron diseases (MND), Huntington’s disease (HD), Spinocerebellar ataxia (SCA), Spinal muscular atrophy (SMA).

### DESCRIPTION

Currently, there are no available therapeutics to prevent or slow down the progression of neurodegenerative diseases, such as Alzheimer’s and Parkinson’s. The disclosure relates to a chemotype that has been shown herein to be a biologically active enhancers of mammalian proteasomes. The chemotype described herein is based on astemizole and derivatives thereof.

The disclosure relates to astemizole and derivates thereof, and their use to, among other things, prevent or slow down the progression of neurodegenerative diseases. The disclosure therefore relates to compounds of the formula (I):

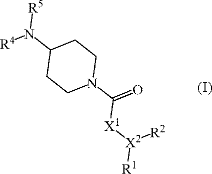

or a pharmaceutically acceptable salt, polymorph, prodrug, solvate or clathrate thereof, wherein:
R¹ is aryl (e.g., phenyl) or heteroaryl (e.g., an indolinyl group or a benzimidazolinyl group);
R² is H, alkyl, aryl or heteroaryl;
X¹ is alkyl (e.g., C₁-C₆ alkyl) or alkenyl;
X² is N or CR³, wherein R³ is absent (e.g., when X¹ is alkenyl), hydrogen, alkyl, heterocyclyl, or aryl;
R⁴ is hydrogen, alkyl, cycloalkyl, heterocyclyl, aryl or heteroaryl; and
R⁵ is hydrogen, alkyl, cycloalkyl, heterocyclyl, aryl or heteroaryl.

Another example of a compound of formula (I) is a compound of the formula (Ia):

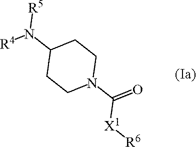

or a pharmaceutically acceptable salt, polymorph, prodrug, solvate or clathrate thereof; wherein:

R⁶ is aryl or heteroaryl (e.g., tricyclic heteroaryl, such as a phenothiazinyl group and a carbazolyl group; and bicyclic heteroaryl, such as an indolinyl group or a benzimidazolinyl group).

Yet another example of a compound of formula (I) are compounds of the formulae (Ib) and (Ic):

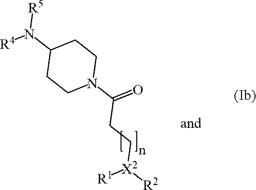

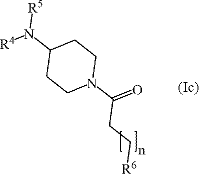

or a pharmaceutically acceptable salt, polymorph, prodrug, solvate or clathrate thereof, wherein:
n is 0, 1 or 2.

Also contemplated herein are compounds of the formulae:

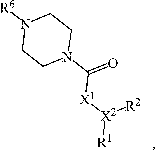

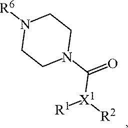

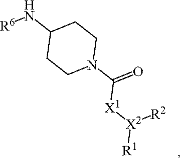

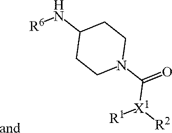

or a pharmaceutically acceptable salt, polymorph, prodrug, solvate or clathrate thereof, wherein:
R¹ is aryl (e.g., phenyl) or heteroaryl (e.g., an indolinyl group or a benzimidazolinyl group);
R² is H, alkyl, aryl or heteroaryl;
X¹ is alkyl or alkenyl;
X² is N or CR³, wherein R³ is absent (e.g., when X¹ is alkenyl), hydrogen, alkyl, heterocyclyl, or aryl; and
R⁶ is aryl or heteroaryl (e.g., tricyclic heteroaryl, such as a phenothiazinyl group and a carbazolyl group; and bicyclic heteroaryl, such as an indolinyl group or a benzimidazolinyl group), such as a compound of the formula:

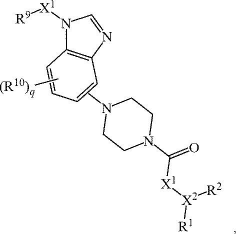

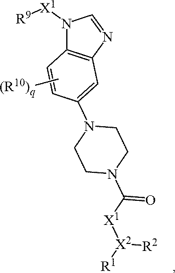

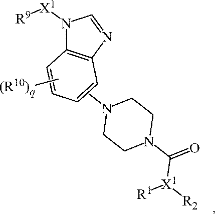

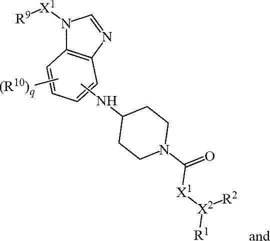

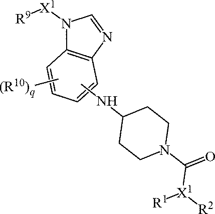

wherein :
   q is 0, 1, 2 or 3;
   R⁹ is alkyl, cycloalkyl aryl, heteroaryl, acyl, amide or carbamate; and
   each R¹⁰ is independently H, halo, alkyl, haloalkyl (e.g., CF₃), alkoxy (e.g., -OR¹¹, wherein R¹¹ is alkyl, cycloalkyl or aryl, heteroaryl, acyl, amide or carbamate) or heterocyclyl (e.g., heteroaryl); or
   two R¹⁰ groups on adjacent carbon atoms, together with those carbon atoms, can form a cycloalkenyl, aryl or heterocyclyl (e.g., heteroaryl); or
   R⁹ and an R¹⁰, together with the atoms to which they are attached, can form a heterocylyl group.

In the compounds of the formulae (I) and (Ia)-(Ic), R¹ can be aryl, such as substituted aryl. For example, R¹ can be substituted or unsubstituted phenyl. In addition or alternatively, R² can be H. In addition, or alternatively, X¹ can be alkyl, such as C₁-C₆ alkyl, branched or unbranched, substituted or unsubstiuted.

In the compounds of the formulae (I) and (Ia)-(Ic), the aryl and heteroaryl groups of R¹ can be unsubstituted or substituted as described herein. For example, when the aryl or heteroaryl groups of R¹ are substituted, they can be substituted with halo (e.g., Cl, Br, and F), amino, OR⁷, wherein R⁷ is hydrogen, alkyl, cycloalkyl, aryl or arylalkyl, S(O)ₓ, wherein x is 0, 1 or 2, acyl, amido or heterocyclyl.

In the compounds of the formulae (I) and (Ia)-(Ic), the alkyl, aryl, and heteroaryl groups of R² can be unsubstituted or substituted as described herein. For example, when the alkyl, aryl or heteroaryl groups of R² are substituted, they can be substituted with halo (e.g., Cl, Br, and F), amino, OR⁷, wherein R⁷ is hydrogen, alkyl, cycloalkyl, aryl or arylalkyl, S(O)ₓ, wherein x is 0, 1 or 2, acyl, amido or heterocyclyl.

In the compounds of the formulae (I) and (Ia)-(Ic), the alkyl, cycloalkyl, heterocyclyl, aryl, and heteroaryl groups of R⁴ can be unsubstituted or substituted as described herein. For example, when the alkyl, cycloalkyl, heterocyclyl, aryl or heteroaryl groups of R⁴ are substituted, they can be substituted with alkyl, cycloalkyl, aryl, heteroaryl, halo (e.g., Cl, Br, and F), amino, OR⁷, wherein R⁷ is hydrogen, alkyl, cycloalkyl, aryl or arylalkyl, S(O)ₓ, wherein x is 0, 1 or 2, acyl, amido or heterocyclyl.

In the compounds of the formulae (I) and (Ia)-(Ic), the alkyl, cycloalkyl, heterocyclyl, aryl, and heteroaryl groups of R⁵ can be unsubstituted or substituted as described herein. For example, when the alkyl, cycloalkyl, heterocyclyl, aryl or heteroaryl groups of R⁵ are substituted, they can be substituted with alkyl, cycloalkyl, aryl, heteroaryl, halo (e.g., Cl, Br, and F), amino, OR⁷, wherein R⁷ is hydrogen, alkyl, cycloalkyl, aryl or arylalkyl, S(O)ₓ, wherein x is 0, 1 or 2, acyl, amido or heterocyclyl.

In the compounds of the formulae (I) and (Ia)-(Ic), the aryl or heteroaryl groups of R⁶ can be unsubstituted or substituted as described herein. For example, when the aryl or heteroaryl groups of R⁶ are substituted, they can be substituted with alkyl, cycloalkyl, aryl, heteroaryl, halo (e.g., Cl, Br, and F), amino, OR⁷, wherein R⁷ is hydrogen, alkyl, cycloalkyl, aryl or arylalkyl, S(O)ₓ, wherein x is 0, 1 or 2, acyl, amido or heterocyclyl.

An example of a compound of the formulae (I) and (Ia)-(Ic) includes, but is not limited to, a compound of the formula:

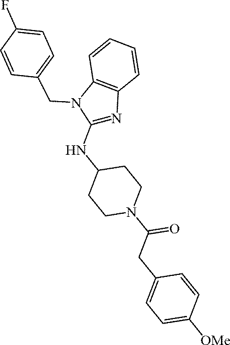 [SMILES: COc1ccc(CC(=O)N2CCC(Nc3nc4ccccc4n3Cc3ccc(F)cc3)CC2)cc1]

and

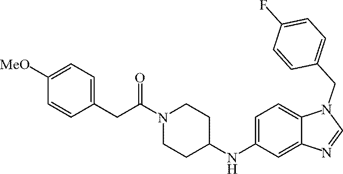 [SMILES: COc1ccc(CC(=O)N2CCC(Nc3ccc4c(c3)ncn4Cc3ccc(F)cc3)CC2)cc1]

pharmaceutically acceptable salts, polymorphs, prodrugs, solvates or clathrates thereof.

This disclosure also relates to compounds of the formula (II) and (IIa)-(IId):

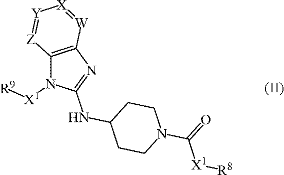

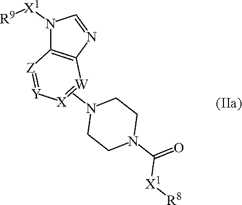

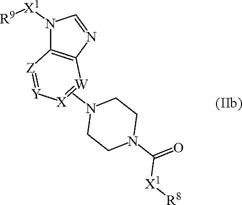

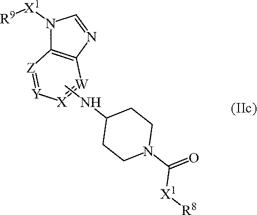

and

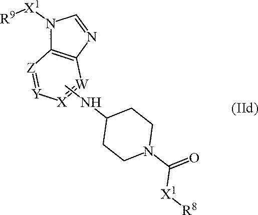

such as

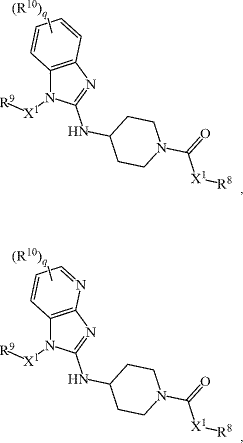

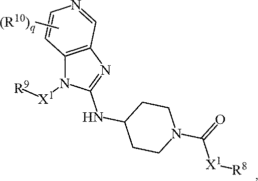

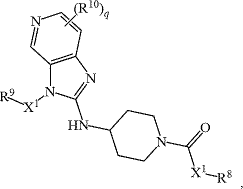

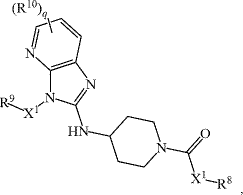

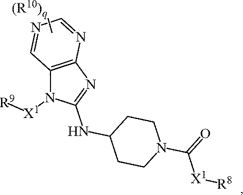

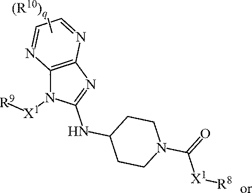

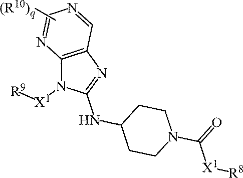

or a pharmaceutically acceptable salt, polymorph, prodrug, solvate or clathrate thereof, wherein:
q is 0, 1, 2 or 3;
each X¹ is independently alkyl (e.g., -(CH₂)ₙ-, wherein n is 0, 1, or 2) or alkenyl;
R⁸ and R⁹ are each independently alkyl, cycloalkyl aryl, heteroaryl, acyl, amide or carbamate; and
W is N or C-R¹⁰; X is N or C-R¹⁰; Y is N or C-R¹⁰; and Z is N or C-R¹⁰; wherein each R¹⁰ is independently H, halo, alkyl, haloalkyl (e.g., CF₃), alkoxy (e.g., -OR¹¹, wherein R¹¹ is alkyl, cycloalkyl or aryl, heteroaryl, acyl, amide or carbamate) or heterocyclyl (e.g., heteroaryl); or
two R¹⁰ groups on adjacent carbon atoms, together with those carbon atoms, can form a cycloalkenyl, aryl or heterocyclyl (e.g., heteroaryl); or
R⁹ and an R¹⁰, together with the atoms to which they are attached, can form a heterocylyl group.

In one example, in the compounds of the formula (II), R⁹ and R¹⁰, together with the atoms to which they are attached, can form a heterocylyl group such as:

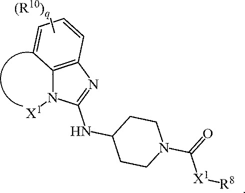

Another variation on compounds of the formula (II) include compounds of the formula:

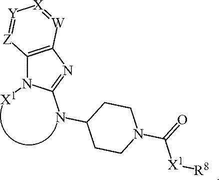

This disclosure also relates to compounds of the formula (III):

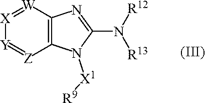

such as

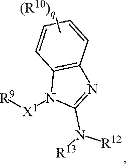

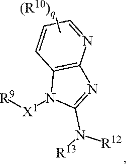

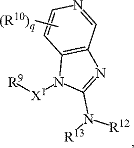

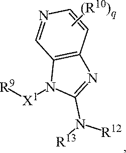

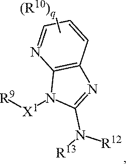

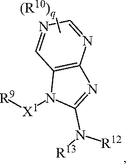

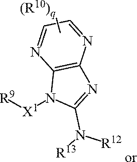

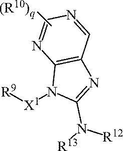

or a pharmaceutically acceptable salt, polymorph, prodrug, solvate or clathrate thereof, wherein:
q is 0, 1, 2 or 3;
X¹ is alkyl (e.g., —(CH₂)ₙ—) or alkenyl;
W is N or C-R¹⁰; X is N or C-R¹⁰; Y is N or C-R¹⁰; and Z is N or C-R¹⁰; wherein each R¹⁰ is independently H, halo, alkyl, haloalkyl (e.g., CF₃), alkoxy (e.g., -OR¹¹, wherein R¹¹ is alkyl, cycloalkyl or aryl, heteroaryl, acyl, amide or carbamate) or heterocyclyl (e.g., heteroaryl); or
two R¹⁰ groups on adjacent carbon atoms, together with those carbon atoms, can form a cycloalkenyl, aryl or heterocyclyl (e.g., heteroaryl); or
R⁹ and an R¹⁰, together with the atoms to which they are attached, can form a heterocylyl group; and
R¹² and R¹³ are each independently alkyl, cycloalkyl aryl, heteroaryl, acyl, amide or carbamate.

In one example, in the compounds of the formula (III), R⁹ and R¹⁰ or R⁹ and R¹³, together with the atoms to which they are attached, can form a heterocylyl group such as:

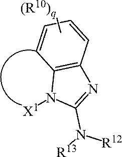

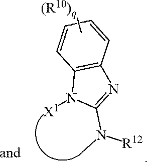

Examples of compounds of the formula (III) include:

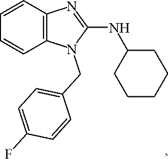

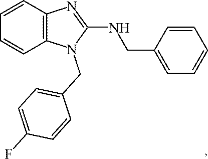

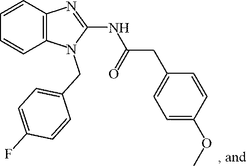

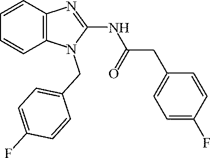 [SMILES: O=C(Cc1ccc(F)cc1)Nc1nc2ccccc2n1Cc1ccc(F)cc1]

or a pharmaceutically acceptable salt, polymorph, prodrug, solvate or clathrate thereof.

This disclosure also relates to compounds of the formula (IV):

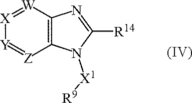

such as

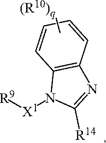

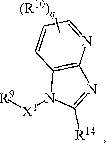

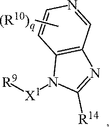

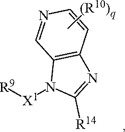

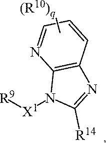

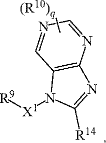

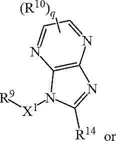

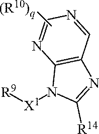

or a pharmaceutically acceptable salt, polymorph, prodrug, solvate or clathrate thereof, wherein:
q is 0, 1, 2 or 3;
X¹ is alkyl (e.g., -(CH₂)ₙ-) or alkenyl;
W is N or C-R¹⁰; X is N or C-R¹⁰; Y is N or C-R¹⁰; and Z is N or C-R¹⁰; wherein each R¹⁰ is independently H, halo, alkyl, haloalkyl (e.g., CF₃), alkoxy (e.g., -OR¹¹ wherein R¹¹ is alkyl, cycloalkyl or aryl, heteroaryl, acyl, amide or carbamate) or heterocyclyl (e.g., heteroaryl); or
two R¹⁰ groups on adjacent carbon atoms, together with those carbon atoms, can form a cycloalkenyl, aryl or heterocyclyl (e.g., heteroaryl); or
R⁹ and an R¹⁰, together with the atoms to which they are attached, can form a heterocylyl group; and
R¹⁴ is aryl (e.g., phenyl) or heterocyclyl (e.g., heteroaryl); or
R⁹ and R¹⁴, together with the atoms to which they are attached, can forma an aryl or a hetorcyclyl.

In one example, in the compounds of the formula (IV), R⁹ and R¹⁰ or R⁹ and R¹⁴, together with the atoms to which they are attached, can form a heterocylyl group such as:

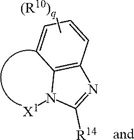

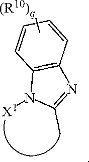

This disclosure also relates to compounds of the formula (V):

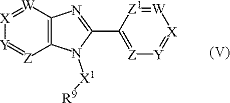

such as

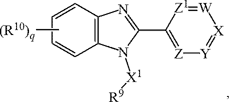

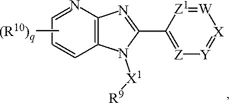

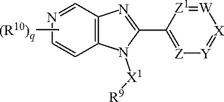

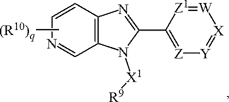

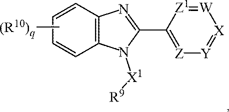

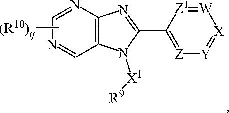

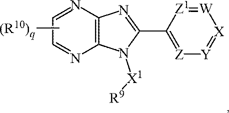

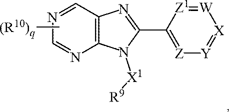

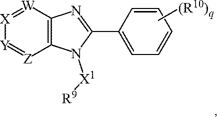

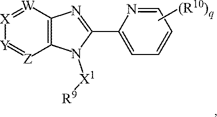

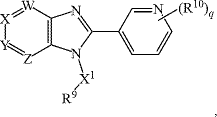

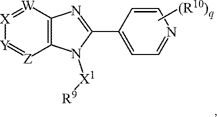

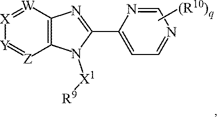

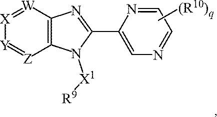

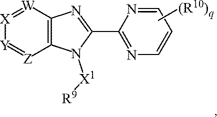

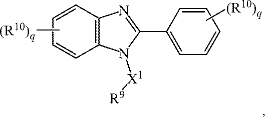

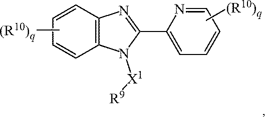

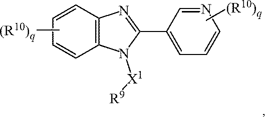

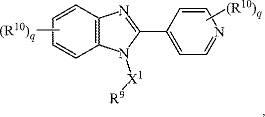

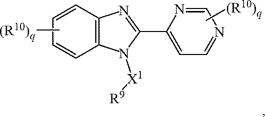

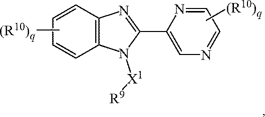

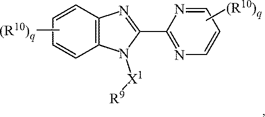

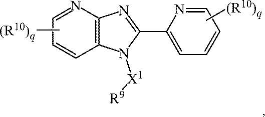

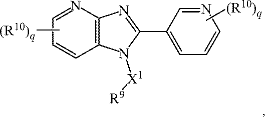

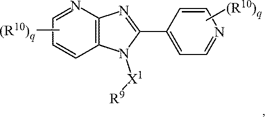

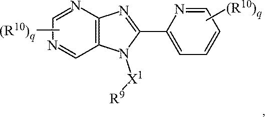

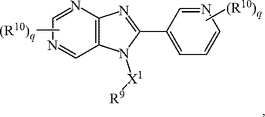

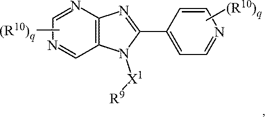

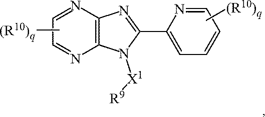

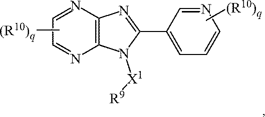

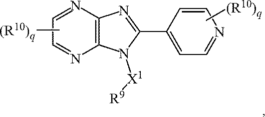

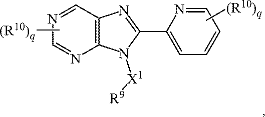

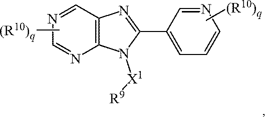

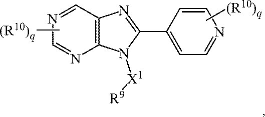

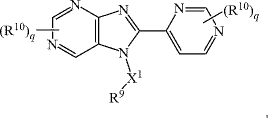

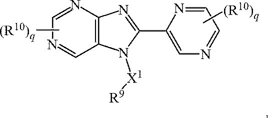

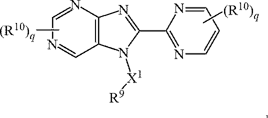

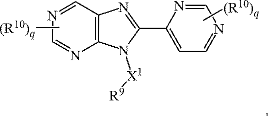

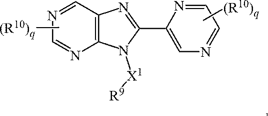

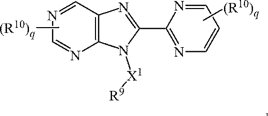

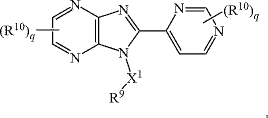

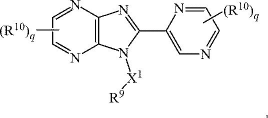

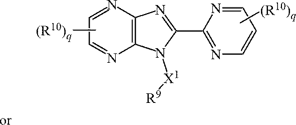

or a pharmaceutically acceptable salt, polymorph, prodrug, solvate or clathrate thereof, wherein:
q is 0, 1, 2 or 3;
X¹ is alkyl (e.g., -(CH₂)ₙ-) or alkenyl;
Z¹ is absent, N or C-R¹⁰;
each W is N or C-R¹⁰; each X is N or C-R¹⁰; each Y is N or C-R¹⁰; and each Z is N or C-R¹⁰; wherein each R¹⁰ is independently H, halo, alkyl, haloalkyl (e.g., CF₃), alkoxy (e.g., -OR¹¹, wherein R¹¹ is alkyl, cycloalkyl or aryl, heteroaryl, acyl, amide or carbamate) or heterocyclyl (e.g., heteroaryl); and
two R¹⁰ groups on adjacent carbon atoms, together with those carbon atoms, can form a cycloalkenyl, aryl or heterocyclyl (e.g., heteroaryl); or
R⁹ and an R¹⁰, together with the atoms to which they are attached, can form a heterocylyl group.

In one example, in the compounds of the formula (V), R⁹ and R¹⁰, together with the atoms to which they are attached, can form a heterocylyl group such as:

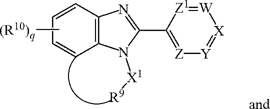

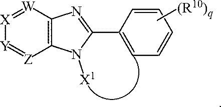

An example of a compound of the formula (V) includes:

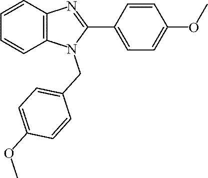 [SMILES: COc1ccc(Cn2c(-c3ccc(OC)cc3)nc3ccccc32)cc1]

or a pharmaceutically acceptable salt, polymorph, prodrug, solvate or clathrate thereof.

Examples of compounds of the formulae (V) include:

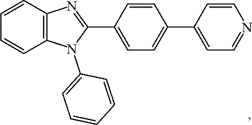

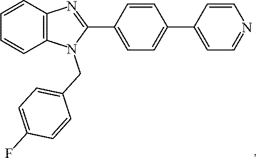

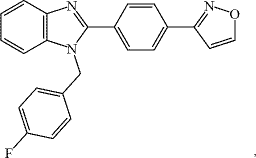

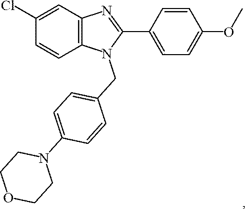

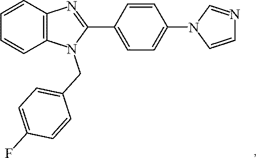

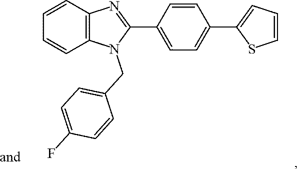

or a pharmaceutically acceptable salt, polymorph, prodrug, solvate or clathrate thereof.

Compounds of the disclosure can have predicted/calculated binding affinities to the 20S proteasome as follows:

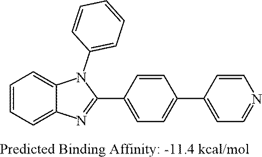

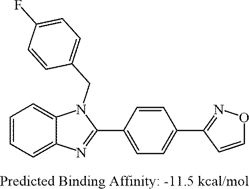

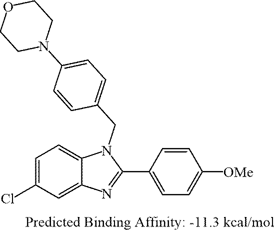

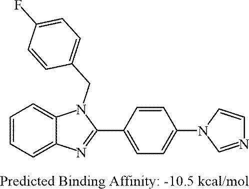

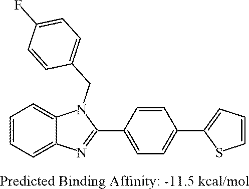

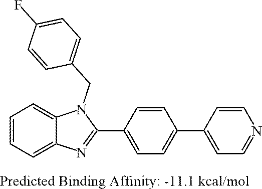

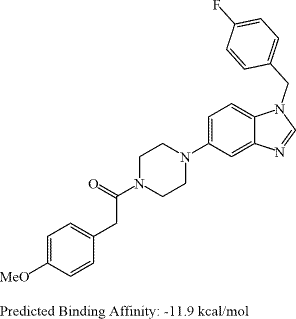

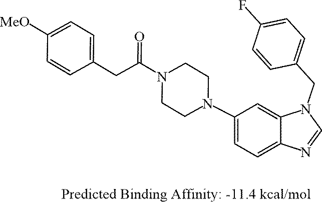

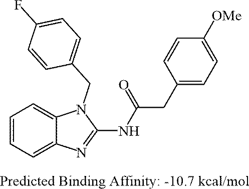

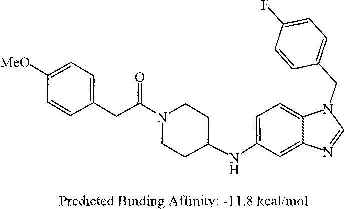

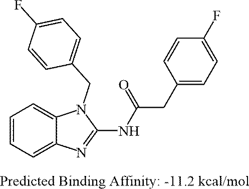

This disclosure also contemplates pharmaceutical compositions comprising one or more compounds and one or more pharmaceutically acceptable excipients. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carrier, sometimes a liquid, in which an active therapeutic agent is formulated. The excipient generally does not provide any pharmacological activity to the formulation, though it can provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes, but is not limited to, any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the present invention can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions of the present invention can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the present invention can vary according to factors such as the disease state, age, gender, weight, patient history, risk factors, predisposition to disease, administration route, pre-existing treatment regime (e.g., possible interactions with other medications), and weight of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in subjects. In therapeutic use for treatment of conditions in mammals (e.g., humans) for which the compounds of the present invention or an appropriate pharmaceutical composition thereof are effective, the compounds of the present invention can be administered in an effective amount. The dosages as suitable for this invention can be a composition, a pharmaceutical composition or any other compositions described herein.

For each of the recited embodiments, the dosage is typically administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition of this invention can be to effect prophylaxis of recurring symptoms. For example, the dosage can be administered once or twice a day to prevent the onset of symptoms in patients at risk, especially for asymptomatic patients.

The absolute weight of a given compound included in a unit dose for administration to a subject can vary widely. For example, about 0.0001 to about 1 g, or about 0.001 to about 0.5 g, of at least one compound of this disclosure, or a plurality of compounds can be administered. Alternatively, the unit dosage can vary from about 0.001 g to about 2 g, from about 0.005 g to about 0.5 g, from about 0.01 g to about 0.25 g, from about 0.02 g to about 0.2 g, from about 0.03 g to about 0.15 g, from about 0.04 g to about 0.12 g, or from about 0.05 g to about 0.1 g.

Daily doses of the compounds can vary as well. Such daily doses can range, for example, from about 0.01 g/day to about 10 g/day, from about 0.02 g/day to about 5 g/day, from about 0.03 g/day to about 4 g/day, from about 0.04 g/day to about 3 g/day, from about 0.05 g/day to about 2 g/day, and from about 0.05 g/day to about 1 g/day.

It will be appreciated that the amount of compound(s) for use in treatment will vary not only with the particular carrier selected but also with the route of administration, the nature of the condition being treated, and the age and condition of the patient. Ultimately the attendant health care provider may determine proper dosage.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

The compositions can include the compounds described herein in a “therapeutically effective amount.” Such a therapeutically effective amount is an amount sufficient to obtain the desired physiological effect, such as a reduction of at least one symptom of cancer or an inflammatory disease or condition.

The compositions contemplated herein can contain other ingredients such as chemotherapeutic agents, anti-inflammatory agents, anti-viral agents, antibacterial agents, antimicrobial agents, immunomodulatory drugs, such as lenalidomide, pomalidomide or thalidomide, histone deacetylase inhibitors, such as panobinostat, preservatives or combinations thereof.

This disclosure also includes methods for treating neurodegenerative diseases, such as Parkinson’s disease, Alzheimer’s disease, Huntington’s disease, and ALS, comprising administering a therapeutically effective amount of at least one of the compounds described herein (e.g., astemizole or compounds of the formulae (I), (Ia)-(Ic), (II), (IIa)-(IId), (III)-(V), and (Va)) to a subject in need thereof. This disclosure also includes methods for reducing, substantially eliminating or eliminating dysregulation of proteostasis comprising administering a therapeutically effective amount of at least one of the compounds described herein (e.g., astemizole or compounds of the formulae (I), (Ia)-(Ic), (II)-(V), and (Va)) to a subject in need thereof. This disclosure also includes methods for reducing, substantially eliminating or eliminating the accumulation of intrinsically disordered proteins (e.g., α-syn) comprising administering a therapeutically effective amount of at least one of the compounds described herein (e.g., astemizole or compounds of the formulae (I), (Ia)-(Ic), (II)-(V), and (Va)) to a subject in need thereof.

As used herein, the terms “treat” and “treating” are not limited to the case where the subject (e.g. patient) is cured and the disease is eradicated. Rather, treatment that merely reduces symptoms, and/or delays disease progression is also contemplated.

The pharmaceutical compositions disclosed herein can have the ability to effectively treat new patient segments where proteasome inhibition and reduced toxicity is desired or warranted.

The compounds and methods described herein can be used prophylactically or therapeutically. The term “prophylactic” or “therapeutic” treatment refers to administration of a drug to a host before or after onset of a disease or condition. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom). Administering the compounds described herein (including enantiomers and salts thereof) is contemplated in both a prophylactic treatment (e.g. to patients at risk for disease, such as elderly patients who, because of their advancing age, are at risk for arthritis, cancer, and the like) and therapeutic treatment (e.g. to patients with symptoms of disease or to patients diagnosed with disease).

The term “therapeutically effective amount” as used herein, refers to that amount of one or more compounds of the various examples of the present invention that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂₋. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be -CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl. The term “alkyl” also generally refers to alkyl groups that can comprise one or more heteroatoms in the carbon chain. Thus, for example, “alkyl” also encompasses groups such as —[(CH₂)ₚO]_{q}H and the like.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

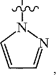

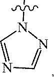

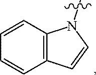

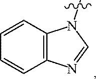

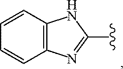

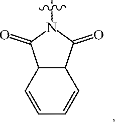

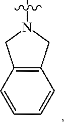

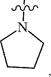

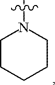

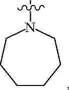

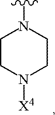

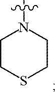

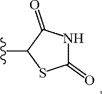

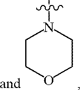

wherein X⁴ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amine” and “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to an nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “amido” as used herein refers to a group having the formula C(O)NRR, wherein R is defined herein and can each independently be, e.g., hydrogen, alkyl, aryl or each R, together with the nitrogen atom to which they are attached, form a heterocyclyl group.

The term “alkylthio” as used herein refers to a sulfur atom connected to an alkyl, alkenyl,or alkynyl group as defined herein.

The term “arylthio” as used herein refers to a sulfur atom connected to an aryl group as defined herein.

The term “alkylsulfonyl” as used herein refers to a sulfonyl group connected to an alkyl, alkenyl,or alkynyl group as defined herein.

The term “alkylsulfinyl” as used herein refers to a sulfinyl group connected to an alkyl, alkenyl, or alkynyl group as defined herein.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” as used herein refers to an amido group connected to two alkyl groups, as defined herein, and which can optionally be linked together to form a ring with the nitrogen. This term also includes the group where the nitrogen is further connected to one or two alkenyl groups in place of the alkyl groups.

The term “substituted” as used herein refers to a group that is substituted with one or more groups including, but not limited to, the following groups: halogen (e.g., F, Cl, Br, and I), R, OR, ROH (e.g., CH₂OH), OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R. C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(=NH)N(R)₂, C(O)N(OR)R, or C(=NOR)R wherein R can be hydrogen, (C₁-C₂₀)alkyl, (C₆-C₂₀)aryl, heterocyclyl or polyalkylene oxide groups, such as polyalkylene oxide groups of the formula —(CH₂CH₂O)₁—R—OR, —(CH₂CH₂CH₂O)_{g}—R—OR, —(CH₂CH₂O)_{f}(CH₂CH₂CH₂O)_{g}—R—OR each of which can, in turn, be substituted or unsubstituted and wherein f and g are each independently an integer from 1 to 50 (e.g., 1 to 10, 1 to 5, 1 to 3 or 2 to 5). Substituted also includes a group that is substituted with one or more groups including, but not limited to, the following groups: fluoro, chloro, bromo, iodo, amino, amido, alkyl, hydroxy, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

In some instances, the compounds described herein (e.g., compounds of the formulae (I), (Ia)-(Ic), (II), (IIa)-(IId), (III)-(V), and (Va)) can contain chiral centers. All diastereomers of the compounds described herein are contemplated herein, as well as racemates.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington’s Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger’s Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

Each embodiment described above is envisaged to be applicable in each combination with other embodiments described herein. For example, embodiments corresponding to formula (I) are equally envisaged as being applicable to formulae (Ia)-(If). Likewise, embodiments corresponding to formula (II) are equally envisaged as being applicable to compounds of the formulae (I), (Ia)-(Ic), (IIa)-(IId), (III)-(V), and (Va) and so forth.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

The invention is now described with reference to the following Examples. The following working examples therefore, are provided for the purpose of illustration only and specifically point out certain embodiments of the present invention, and are not to be construed as limiting in any way the remainder of the disclosure. Therefore, the examples should be construed to encompass any and all variations which become evident as a result of the teaching provided herein.

### EXAMPLES

The present disclosure can be better understood by reference to the following examples which are offered by way of illustration. The disclosure is not limited to the examples given herein.

### Introduction

The human proteasome is part of the cellular machinery that regulates protein degradation. Most proteins are degraded by the 26S proteasome via a ubiquitin-dependent mechanism, however intrinsically disordered proteins (unstructured) proteins can also be degraded the 20S isoform of the proteasome via a ubiquitin-independent mechanism. Intrinsically disordered proteins (IDPs) are named for their lack of tertiary structure allowing them to adopt numerous conformations and interact with multiple binding partners. When the synthesis of IDPs outpaces their rate of degradation, they accumulate and induce toxic signaling events that drive many human diseases.

Arguably the most infamous IDP associated with cancer initiation, progression and relapse is the pro-oncogenic transcription factor, c-MYC. Over-expression of c-MYC is the driving force in an astonishing 60-70% of all human cancers including multiple myeloma, histiocytic sarcoma, myeloid leukemia, glioblastoma, melanoma, breast cancer, colon cancer, cervical cancer, small-cell lung carcinoma, and osteosarcoma. Small molecule 20S proteasome activators can reduce c-MYC protein levels and therefore prevent the initiation progression and relapse in c-MYC driven cancers.

The disclosure relates to small molecule 20S proteasome activators of the formulae (I), (Ia)-(Ic), (II)-(V), and (Va) as therapeutic agents to treat amyloidogenic diseases including neurodegenerative diseases and type II diabetes. Neurodegenerative diseases include: Alzheimer’s disease (AD) and other dementias, Parkinson’s disease (PD) and PD-related disorders, Prion disease, Motor neuron diseases (MND), Huntington’s disease (HD), Spinocerebellar ataxia (SCA) and Spinal muscular atrophy (SMA). Overwhelming evidence points towards the accumulation and subsequent oligomerization of intrinsically disordered proteins (IDPs) such as amyloid-b, a-synuclein, polyQ, and dipeptide repeat (DPR) units as the driving causes of these diseases. These soluble oligomeric forms are also responsible for impairing proteasome function, which further drives disease progression. Robust data demonstrates that enhancing proteasome activity prevents the accumulation of IDPs, reduce brain damage, prevent dementia and may be a new therapeutic strategy to treat neurodegenerative diseases.

### Identification

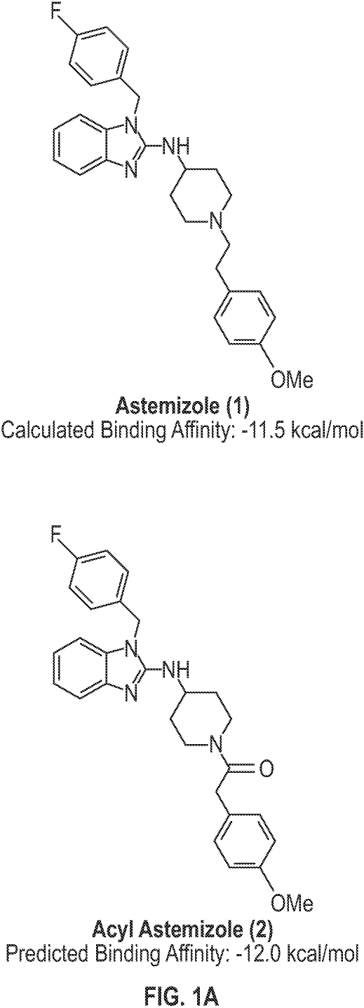

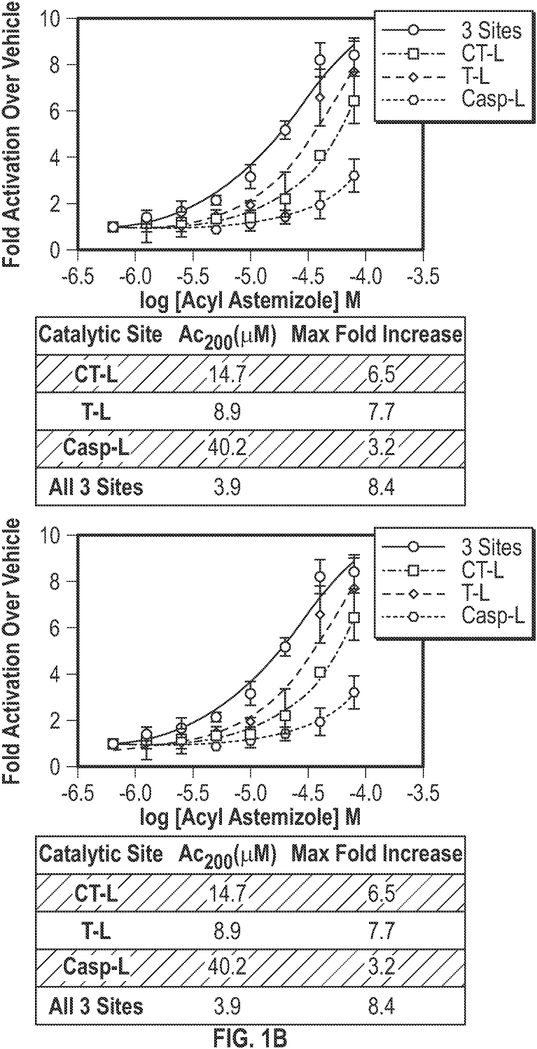

The antihistamine drug astemizole (FIGS. 1A, 1) as a promising new scaffold for the development of 20S activators due to its strong enhancement of 20S proteolysis. To assess astemizole’s 20S proteasome activity, a series of assays were performed using each of three fluorogenic peptide substrates. These substrates were a chymotryptic-like (CT-L), a trypsin-like (T-L) and a caspase-like (Casp-L) substrate, one for each of the catalytic sites of the proteasome. It has been shown that the proteasome’s active sites allosterically regulate each other in the presence of their individual substrates. Therefore, a combination of the three probes to represent the overall activity of a 20S activator more accurately in a system in which all catalytic sites are interacting. Astemizole activates all three catalytic sites of the 20S proteasome (FIG. 1B) and achieved a doubling of activity (hereafter referred to as AC₂₀₀) using the combination of probes at 3.3 µM (i.e. AC₂₀₀ 3.3 µM), with a maximum fold enhancement of nearly 7-fold (i.e. 700%).

### Design of Analogues

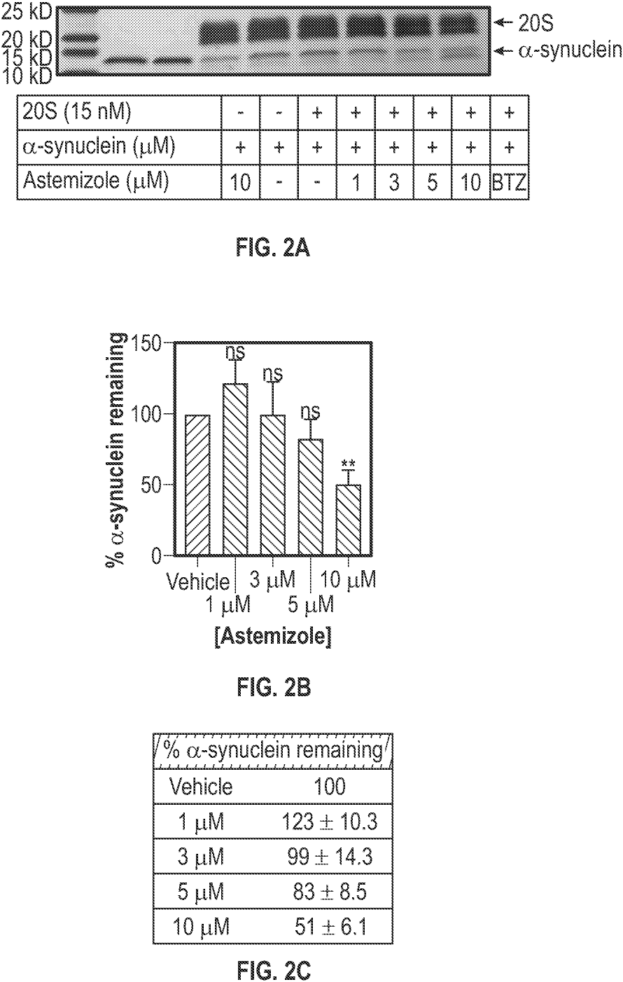

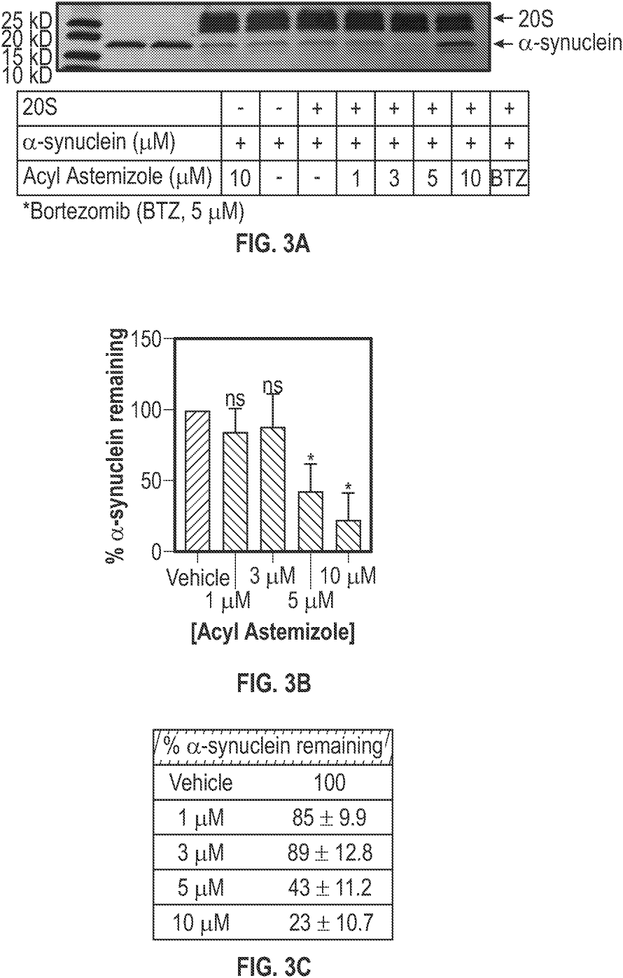

Astemizole is a potent antihistamine as an H1 receptor antagonist. As such, it has good drug-like properties and penetrates the blood brain barrier (BBB) effectively, which makes it a promising scaffold for the development of novel 20S activators. On the other hand, due to its activity and side-effects (QTc interval prolongation and related arrhythmias due to hERG channel blockade) it cannot be repurposed therapeutically without modification. Therefore, structural modifications known to reduce its H1- receptor activity and possible side effects were prepared. N-acylated astemizole (2), was designed to eliminate fluspirilene’s H1 receptor activity. In this scaffold, the basicity of the piperidine’s amine has been reduced through its conversion to an amide. Molecular docking studies were performed using Autodock Vina. Astemizole and its analogues found to preferentially bind to the α1-2 intersubunit pocket (FIG. 2A and FIG. 2B). This mode of binding is different from our previously reported 20S proteasome activators which, when docked, preferentially bind to the α1-2 intersubunit pocket of the 20S proteasome.

### Synthesis

N-acyl astemizole (2) was prepared as described in Scheme 1).

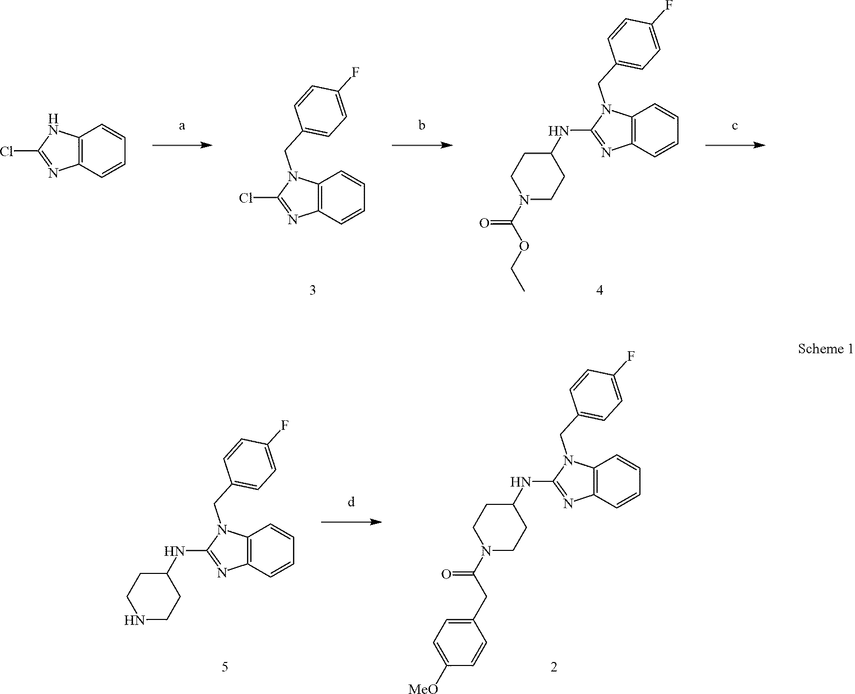

Benzylation of compound 3 was accomplished using KOH and 4-fluorobenzyl bromide. Various substituents can be incorporated into the C-2 position using different nucleophiles. For example, compound 4 was prepared by heating 3 with ethyl 4-aminopiperidinecarboxylate. Deprotection of the ethyl carbamate using HBr provided compound 7. Acylation of the piperidine with 4-methoxyphenylacetyl chloride rendered N-acyl astemizoles (2).

### In Vitro Testing, 20S Activation

The 20S activity of astemizoles and N-acylastemizole (compound 2) was further assessed in the fluorogenic peptide assay, using each of the individual substrates as well as the combination of the three (FIG. 1B). When compared to astemizole, the N-acylated analog performs similarly using the combination of the three peptide substrate probes, with an AC200 of 3.9 µM. The N-acylated analog achieved similar max fold increases for each substrate/combination (>800% increase over vehicle).

### In Vitro Testing, IDP Degradation

Purified human 20S proteasome was incubated with the compounds, astemizoles (1) and N-acylated astemizole (2), at various concentrations, and human α-syn substrate was subsequently added to the mixtures. The resulting digestions were visualized using silver stains. Enhanced 20S activity is measured as a reduction of remaining α-syn when compared to the vehicle control. As shown in FIG. 2A, both astemizole and N-acylated astemizole were able to effectively enhance the degradation of α-syn by the 20S proteasome in vitro. Both compounds displayed a significant (FIG. 2B, >50%, p<0.001) concentration-depended decrease in α-syn at values near their AC200. These results grant confidence in this novel 20S activator scaffold to induce the degradation of IDPs and prevent their accumulation.

### Myc Reporter Assay

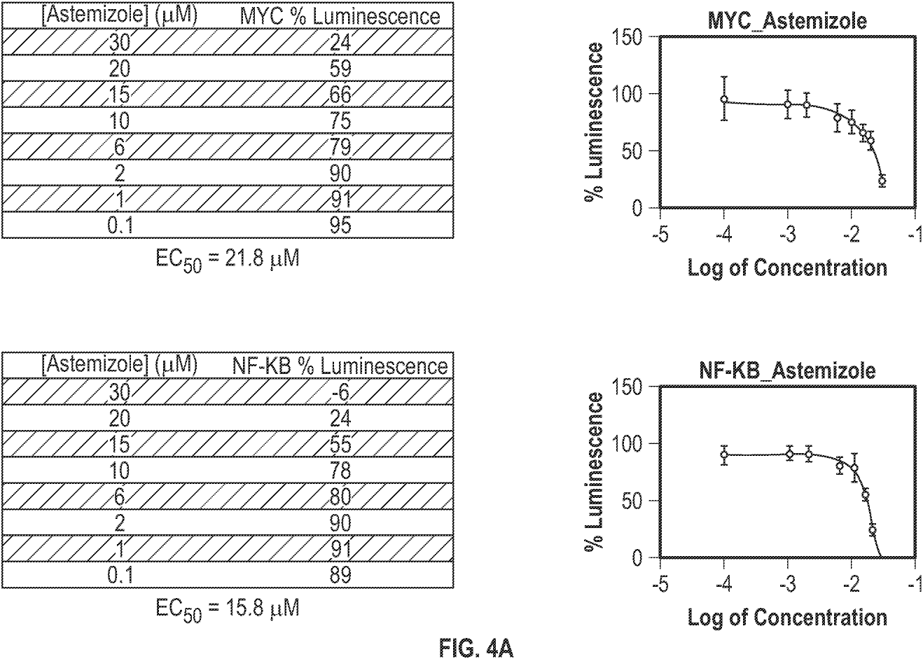

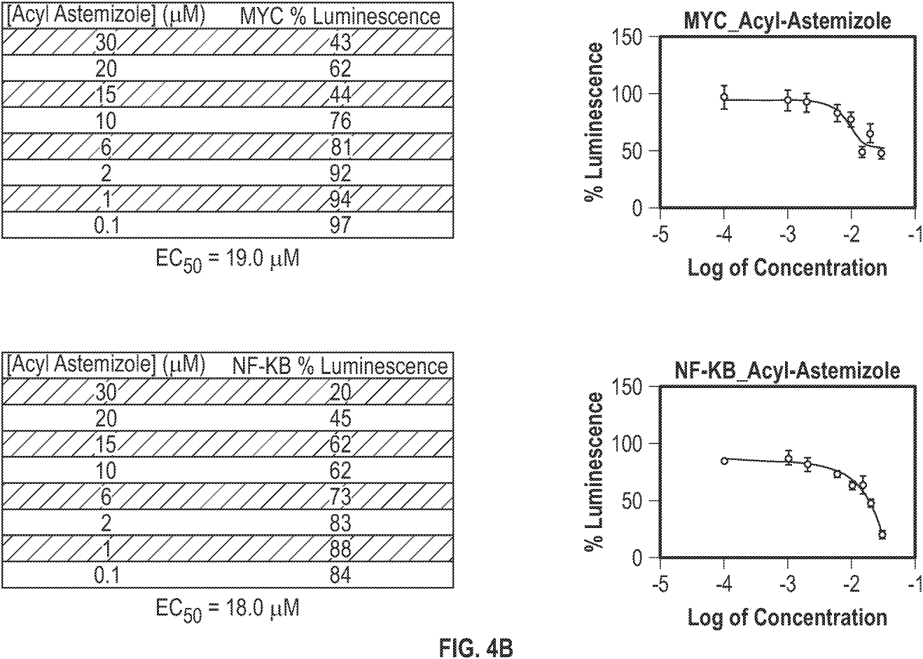

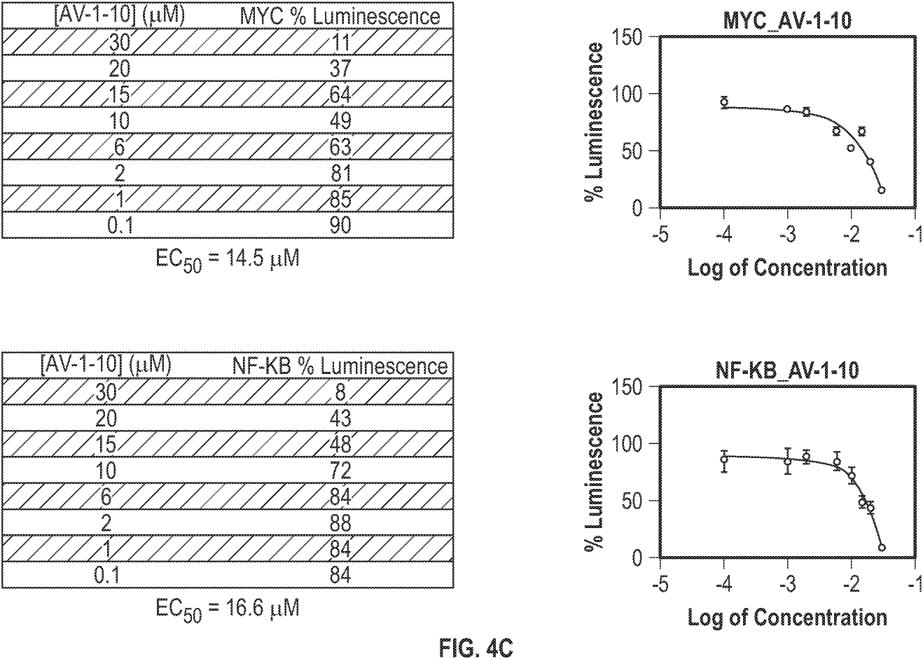

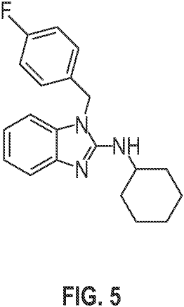

The ability of the compounds to modulate MYC-mediated gene transcription was evaluated using a luciferase reporter assay in HCT116 cells with a stably transfected MYC-luciferase gene. HCT-116 cells stable transfected Myc-Luc reported cells were seeded in a white, clear bottom, 96-well microplate at 25,000 cells/well, in McCoy’s 5A media supplemented with 10% FBS and 1% Penicillin/Streptomycin. The plate was incubated at 37° C. with 5% CO2 overnight. The next day, cells were treated with 0.0 µM, 0.1 µM, 1.0 µM, 2.5 µM, 5.0 µM, 10 µM, or 20 µM TCH-165, in Opti-MEM I supplemented with 0.5% FBS, 1% Non-essential amino acids, 1 mM Sodium Pyruvate and 1% Penicillin/Streptomycin. The plate was incubated at 37° C. with 5% CO2 for 16 hours. Firefly luminescence was measured using the One-Step Luciferase Assay System from BPS Biosciences. Data reported as a n=3 and the IC50 values were calculated using the equation for the sigmoidal curve for variable slope. Compound 1 had an EC50 of 21.8 microM for inhibition of c-MYC and compound 2 had an EC50 of 19 microM. For a dose response see FIG. 4A for compound 1 and FIG. 4B for compound 2.

### NF-κB Reporter Assay

HCT-116 stable transfected NF-kappaB-luc reporter cells were seeded into in a white, clear bottom, 96-well microplate at 5,000 cells/well, in McCoy’s 5A media supplemented with 10% FBS and 1% Penicillin/Streptomycin. The plate was incubated at 37° C. with 5% CO2 overnight. The next day, cells were pretreated with 0.0 µM, 0.1 µM, 1.0 µM, 2.5 µM, 5.0 µM, 10 µM, or 20 µM TCH-165, in Opti-MEM I supplemented with 0.5% FBS, 1% Non-essential amino acids, 1 mM Sodium Pyruvate and 1% Penicillin/Streptomycin, for 1 hour. TNF-alpha (Gibco, REF# PHC3016) was added to a final concentration of 2 ng/mL and the samples were further incubated for 16 hours at 37° C. in 5% CO2. Firefly luminescence was measured using the One-Step Luciferase Assay System from BPS Biosciences. HCT-116 cells stable transfected NF-kappaB-Luc reported cells were exposed to various concentrations of the compounds (0.1-20 microM) for 16 hours after which luminescence was measured and compared to vehicle control. Data reported as a n=3 and the IC50 values were calculated using the equation for the sigmoidal curve for variable slope. Data reported as a n=3 and the IC50 values were calculated using the equation for the sigmoidal curve for variable slope. Compound 1 had an EC50 of 15.8 microM for inhibition of NF-kB transcription and compound 2 had an EC50 of 18 µM. For a dose response see FIG. 4A for compound 1 and FIG. 4B for compound 2.

### Materials and Reagents

Human 20S proteasome and fluorogenic substrates N-succinyl-Leu-Leu-Val-Tyr-7-amido-4-methylcoumarin (Suc-LLVY-AMC), carboxyl benzyl-Leu-Leu-Glu-7-amido-4-methylcoumarin (Z-LLE-AMC), tert-butyloxycarbonyl-Leu-Arg-Arg-7-amido-4-methylcoumarin (Boc-LRR-AMC), and bortezomib were obtained from Boston Biochem, Inc. (Cambridge, MA). The PVDF membrane, Clarity western ECL reagent, blocking grade milk, and precast sodium dodecyl sulfate gels were from Bio-Rad (Hercules, CA). The recombinant wild type α-synuclein was obtained from Abcam (Cambridge, MA). Rabbit polyclonal anti-α-synuclein, mouse monoclonal anti-α-synuclein and goat anti-rabbit HRP-linked antibody were purchased from Santa Cruz Biotechnologies (Dallas, TX). The anti-mouse HRP-linked antibody was purchased from Cell Signaling Technology (Danvers, MA). The α-synuclein aggregates were obtained from Novus Biologicals (Littleton, CO). Unless otherwise noted, chemicals were purchased from commercial suppliers and used without further purification.

### Molecular Docking Studies

Docking was performed using AutoDock Vina, supported through computational resources and services provided by the Institute for Cyber-Enabled Research at Michigan State University. The crystal structure of the closed gate human proteasome (h20S) was obtained from the PDB database (PDB ID: 4R30). Molecules were generated in Perkin Elmer’s Chem3D, minimized using the MM2 force field, and converted to PDB. These molecules were uploaded to PyRx and converted to ligand pdbqt files. Small molecule ligands were then docked against the entirety of the h20S proteasome (grid box 153.2 × 138.0 × 189.4 A) three times with exhaustiveness set to 1000. Individual poses were manually inspected using Pymol and BIOVIA Discovery Studio 2020.

### Fluorogenic Peptide Degradation 20S Proteasome Activity Assay

Activity assays were carried out in a 100 µL reaction volume. Different concentrations (1-80 µM) of test compounds were added to a black flat/clear bottom 96-well plate containing 1 nM of human constitutive 20S proteasome, in 50 mM Tris-HCl at pH 7.8, 100 mM NaCl and allowed to incubate for 15 min at 37° C. Fluorogenic substrates were then added and the enzymatic activity measured at 37° C. on a SpectraMax M5e spectrometer by measuring the change in fluorescence unit per minute for 1 hour at 380-460 nm. The fluorescence units for the vehicle control were set at a 100%, and the ratio of drug-treated sample set to that of vehicle control was used to calculate the fold change in enzymatic activity. The fluorogenic substrates used were one of the following: Suc-LLVY-AMC (CT-L activity, 20 µM), Z-LLE-AMC (Casp-L activity, 20 µM), Boc-LRR-AMC (T-L activity, 40 µM) or a combination of the three substrates each at 6.67 µM.

### General Experimental Information

Reactions were carried out under a nitrogen atmosphere in flame-dried glassware. Solvents and reagents were purchased from commercial suppliers and used without further purification. Anhydrous THF was distilled over sodium and benzophenone directly before use. Magnetic stirring was used for all reactions. Yields refer to chromatographically and spectroscopically pure compounds unless otherwise noted. Infrared spectra were recorded on a Jasco Series 6600 FTIR spectrometer. ¹H and 13C NMR spectra were recorded on a Varian Unity Plus-500 or 600 spectrometers. Chemical shifts are reported relative to the residue peaks of the solvent (CDCl₃: 7.26 ppm for 1H and 77.0 ppm for 13C) (DMSO-d₆: 2.50 ppm for ¹H and 39.5 ppm for ¹³C). The following abbreviations are used to denote the multiplicities: s = singlet, d = doublet, dd = doublet of doublets, t = triplet, and m = multiplet. HRMS were obtained at the Mass Spectrometry Facility of Michigan State University with a Micromass Q-ToF Ultima API LC-MS/MS mass spectrometer.

### In Vitro Purified Α-Synuclein Degradation Assay (Silver Stain)

Digestion of α-synuclein was carried out in a 50 µL reaction volume made of 50 mM Tris, 100 mM NaCl at pH 7.8; 0.5 µM purified α-synuclein and 15 nM purified human 20S proteasome. Briefly, 20S proteasome was diluted to 17 nM in the reaction buffer. Test compounds or vehicle (1 µL of 50× stock or DMSO) were added to 44 µL of 7.58 nM 20S and incubated at 37° C. for 15 min. 5 µL of 5 µM α-synuclein substrate was then added to the reaction mixture and incubated at 37° C. for 2 hours. The reactions were quenched with concentrated sodium dodecyl sulfate (SDS) loading buffer. After boiling for 10 min, samples were resolved on a 4-20% Tris-glycine SDS-PAGE gel. The gels were then stained using a Pierce Silver Stain Kit (Thermo Scientific, Rockford IL) and the provided procedure.

### MYC Reporter Assay

Myc reporter (Luc) HCT-116 cells were seeded in a white, clear bottom, 96-well microplate at 25,000 cells/well, in McCoy’s 5A media supplemented with 10% FBS and 1% Penicillin/Streptomycin. The plate was incubated at 37° C. with 5% CO₂ overnight. The next day, cells were treated with 0.0 µM, 0.1 µM, 1.0 µM, 2.5 µM, 5.0 µM, 10 µM, or 20 µM TCH-165, in Opti-MEM supplemented with 0.5% FBS, 1% Non-essential amino acids, 1 mM Sodium Pyruvate and 1% Penicillin/Streptomycin. The plate was incubated at 37° C. with 5% CO₂ for 16 hours. Firefly luminescence was measured using the One-Step Luciferase Assay System from BPS Biosciences.

### Example 1

2-Chloro-1-(4-fluorobenzyl)-1H-benzimidazole (3) 2-Chloro-1H-benzimidazole (13.12 mmol, 2 g), KOH (18.37 mmol, 1.03 g) and dry acetonitrile (80 mL) was heated to reflux and stirred for 1 hour. 4-fluorobenzyl bromide (19.68 mmol, 2.45 mL) was added. The reaction mixture was refluxed while stirring for 6 hours. The reaction mixture was cooled to room temperature, washed with water, and extracted with dichloromethane. The organic layer was evaporated and recrystallized with dichloromethane and hexane to afford Compound 2 (2.6 g, 77%) as a white solid.

¹H NMR (500 MHz, Acetone-d₆) δ 7.68 -7.59 (m, 1H), 7.58 -7.50 (m, 1H), 7.39 -7.30 (m, 2H), 7.31-7.23 (m, 2H), 7.19 - 7.07 (m, 2H), 5.55 (d, J = 1.1 Hz, 2H). ¹³C NMR (126 MHz, cd₃od) δ 163.87 (d, J= 245.6 Hz), 142.30, 141.86, 136.22, 132.89, 130.17, 130.11, 124.93, 124.39, 119.58, 116.85, 116.67, 111.66, 48.04. IR (neat): 1602, 1506, 1233 cm⁻¹. m/z: [(M+H)+] calcd for (C₁₄H₁₁ClFN₂⁺) 261.0595; Found 261.0599. mp 70-74° C.

### Example 2

Ethyl 4-((1-(4-fluorobenzyl)-1H-benzimidazole-2-yl)amino) piperidine-1-caryboxylate (4) Compound 3 (0.38 mmol, 100 mg) was dissolved in dimethylacetamide (3 mL), followed by addition of ethyl 4-aminopiperidinecarboxylate (3.8 mmol, 0.65 mL). The reaction mixture was stirred at 155° C. in a silicone oil bath for 68 hours and then cooled to room temperature. The reaction mixture was then diluted with ethyl acetate and poured into a 50% saturated brine solution and then extracted with dichloromethane. The crude product was purified by column chromatography on silica gel (hexane/ethyl acetate; 10:90) to afford Compound 4 (69.5 mg, 45%) as a yellow solid.

¹H NMR (500 MHz, Methanol-d₄) δ 7.35 - 7.26 (m, 1H), 7.15 - 7.09 (m, 2H), 7.06 - 6.98 (m, 4H), 6.93 (ddd, J = 8.1, 7.3, 1.1 Hz, 1H), 5.22 (s, 2H), 4.10 (qd, J = 7.0, 4.0 Hz, 4H), 3.95 (tt, J = 11.0, 4.0 Hz, 2H), 2.98 (s, 1H), 2.10 - 1.98 (m, 2H), 1.45 (dd, J = 11.9, 4.2 Hz, 2H), 1.24 (td, J = 7.1, 3.3 Hz, 3H). ¹³C NMR (126 MHz, Methanol-d₄) δ 163.56 (d, J = 244.6 Hz), 157.19, 155.18, 142.92, 135.30, 133.75, 129.56, 122.50, 120.86, 116.52, 116.35, 116.08, 109.13, 62.70, 51.43, 45.24, 44.10, 33.11, 14.96. IR (neat): 3275, 1694, 1220 cm⁻¹. m/z: [(M+H)+] calcd for (C₂₂H₂₆FNO₂⁺) 397.2040; Found 397.2070. mp 120-123° C.

### Example 3

1-(4-fluorobenzyl)-N-(piperidin-4-yl)-1H-benzo[d]imidazol-2-amine (5) Compound 4 (0.76 mmol, 300 mg) was dissolved in 48% HBr (10 mL) and heated to reflux and stirred for 3 hours. The reaction mixture was cooled to room temperature and neutralized with solid sodium bicarbonate, washed with water, and then extracted with dichloromethane. The organic layer was evaporated to afford Compound 5 (204 mg, 83%) as a yellow solid.

¹H NMR (500 MHz, Chloroform-d) δ 7.46 (d, J = 7.9 Hz, 1H), 7.12 -7.04 (m, 3H), 7.02 - 6.93 (m, 4H), 5.03 (s, 2H), 4.23 (s, 1H), 3.98 (s, 1H), 2.98 (dt, J = 12.9, 3.7 Hz, 2H), 2.68 (td, J = 12.1, 2.6 Hz, 3H), 2.09 -2.02 (m, 2H), 1.33 - 1.25 (m, 2H). ¹³C NMR (126 MHz, Chloroform-d) δ 162.36 (d, J = 247.0 Hz), 153.31, 142.32, 134.47, 131.23, 128.24, 128.18, 121.51, 119.73, 116.39, 116.15, 115.97, 107.25, 50.11, 45.14, 44.93, 33.60. IR (neat): 3290, 3245, 1218 cm⁻¹. m/z: [(M+H)+] calcd for (C₁₉H₂₂FN₄⁺) 325.1828; Found 325.1826. mp 120-126° C.

### Example 4

1-(4-((1-(4-fluorobenzyl)-1H-benzo[d]imidazol-2-yl)amino)piperidin-1-yl)-2-(4-methoxyphenyl)ethan-1-one (2) Compound 5 (0.15 mmol, 50 mg) and triethylamine (0.2 mmol, 0.03 mL) were dissolved in dichloromethane (2 mL), followed by dropwise addition of 4-methoxyphenylacetyl chloride (0.15 mmol, 0.023 mL). The reaction mixture was stirred at room temperature for 4 hours. The crude product was purified by column chromatography (methanol/ethyl acetate 5:95) to afford Compound 2 (31.5 mg, 43%) as a colorless oil.

¹H NMR (500 MHz, Methanol-d₄) δ 7.32 (d, J = 7.8 Hz, 1H), 7.20 - 7.16 (d, 2H), 7.13 - 7.08 (m, 2H), 7.07 - 6.99 (m. 4H), 6.95 (t, J = 7.6 Hz, 1H), 6.91 - 6.84 (d, 2H), 5.49 (s, 1H), 5.22 (s, 2H), 4.52 (dq, J = 13.4, 3.2, 2.8 Hz, 1H), 4.06 - 3.94 (m, 2H), 3.76 (s, 3H), 3.74 (d, J = 4.5 Hz, 1H), 3.68 (d, J = 14.9 Hz, 1H), 3.20 (ddd, J = 14.2, 11.9, 2.6 Hz, 1H), 2.85 (td, J = 12.8, 12.1, 2.7 Hz, 1H), 2.08- 2.02 (d, 1H), 1.99 (d, J= 12.9 Hz, 1H), 1.45 - 1.37 (m, 1H), 1.19 (ddd, J= 13.8, 7.8, 3.3 Hz, 1H). ¹³C NMR (126 MHz, Methanol-d₄) δ 172.32, 163.57 (d, J = 244.4 Hz), 160.06, 142.46, 133.67 (d, J = 3.2 Hz), 130.67, 129.58 (d, J = 8.2 Hz), 128.29, 122.60, 121.03, 116.44 (d, J = 21.9 Hz), 115.98, 115.18, 109.25, 55.64, 51.37, 46.46, 45.27, 42.26, 40.64, 33.52, 32.81. IR (neat): 3289, 1682, 1260 cm⁻¹. m/z: [(M+H)+] calcd for (C₂₈H₃₀FN₄O₂⁺) 473.2353; Found 473.2353.

The disclosure provides for the following example embodiments, the numbering of which is not to be construed as designating levels of importance:

Embodiment 1 relates to a compound of the formula (l):

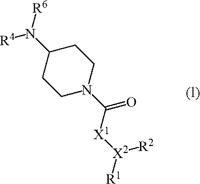

or a pharmaceutically acceptable salt, polymorph, prodrug, solvate or clathrate thereof, wherein:
R¹ is aryl or heteroaryl;
R² is H, alkyl, aryl or heteroaryl;
X¹ is alkyl or alkenyl;
X² is N or CR³, wherein R³ is absent, hydrogen, alkyl, heterocyclyl, or aryl;
R⁴ is hydrogen, alkyl, cycloalkyl, heterocyclyl, aryl or heteroaryl; and
R⁵ is hydrogen, alkyl, cycloalkyl, heterocyclyl, aryl or heteroaryl.

Embodiment 2 relates to the compound of Embodiment 1, wherein R¹ is aryl.

Embodiment 3 relates to the compound of Embodiments 1-2, wherein R¹ is substituted aryl.

Embodiment 4 relates to the compound of Embodiments 1-3, wherein R¹ is substituted phenyl.

Embodiment 5 relates to the compound of Embodiment 1, wherein R¹ is heteroaryl.

Embodiment 6 relates to the compound of Embodiment 1, wherein R¹ is benzimidazolinyl.

Embodiment 7 relates to the compound of Embodiments 1-6, wherein R² is H.

Embodiment 8 relates to the compound of Embodiments 1-7, wherein X¹ is alkyl.

Embodiment 9 relates to the compound of Embodiments 1-8, wherein X¹ is C₁-C₆ alkyl.

Embodiment 10 relates to the compound of Embodiments 1-9, wherein the compound of formula (l) is a compound of the formula (la):

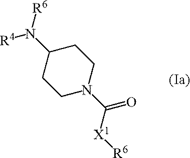

or a pharmaceutically acceptable salt, polymorph, prodrug, solvate or clathrate thereof; wherein:
R⁶ is aryl or heteroaryl.

Embodiment 11 relates to the compound of Embodiments 1-10, wherein the compound of formula (I) is a compound of the the formula (Ib) or (Ic):

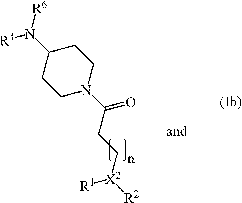

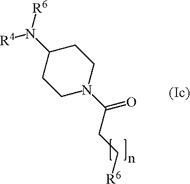

or a pharmaceutically acceptable salt, polymorph, prodrug, solvate or clathrate thereof, wherein:
n is 0, 1 or 2.

Embodiment 12 relates to the compound of Embodiments 1-11, wherein the compound of formula (I) is a compound of the formula:

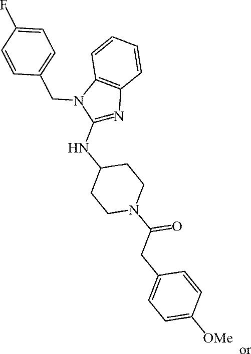

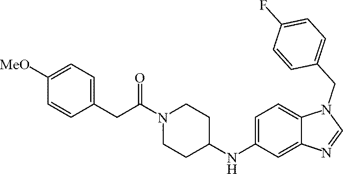 [SMILES: COc1ccc(CC(=O)N2CCC(Nc3ccc4c(c3)ncn4Cc3ccc(F)cc3)CC2)cc1]

pharmaceutically acceptable salts, polymorphs, prodrugs, solvates or clathrates thereof.

Embodiment 13 relates to a compound of the formula:

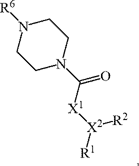

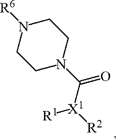

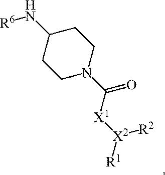

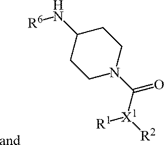

or a pharmaceutically acceptable salt, polymorph, prodrug, solvate or clathrate thereof, wherein:
R¹ is aryl or heteroaryl;
R² is H, alkyl, aryl or heteroaryl;
X¹ is alkyl or alkenyl;
X² is N or CR³, wherein R³ is absent, hydrogen, alkyl, heterocyclyl, or aryl; and
R⁶ is aryl or heteroaryl.

Embodiment 14 relates to the compound of Embodiment 13, wherein the compound is a compound of the formula:

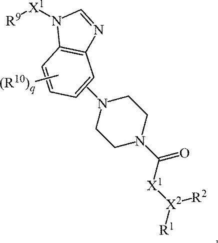

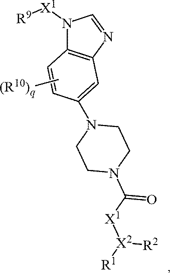

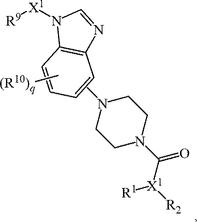

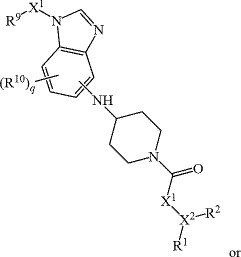

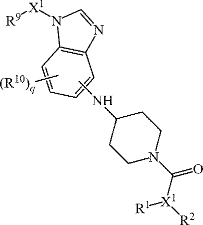

wherein :
q is 0, 1, 2 or 3;
R⁹ is alkyl, cycloalkyl aryl, heteroaryl, acyl, amide or carbamate; and
each R¹⁰ is independently H, halo, alkyl, haloalkyl, alkoxy or heterocyclyl; or
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group.

Embodiment 15 relates to a compound of the formula (II) and (lla)-(lld):

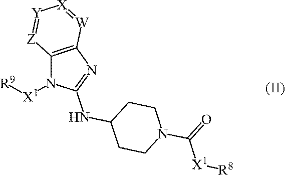

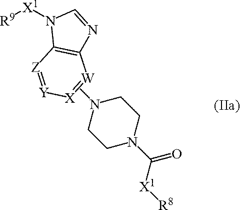

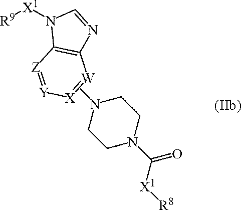

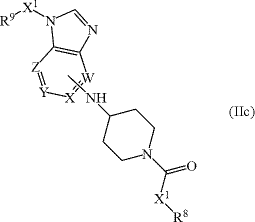

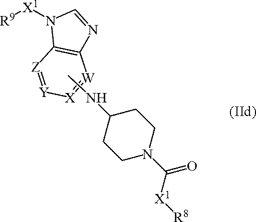

or a pharmaceutically acceptable salt, polymorph, prodrug, solvate or clathrate thereof, wherein:
q is 0, 1, 2 or 3;
each X¹ is independently alkyl or alkenyl;
R⁸ and R⁹ are each independently alkyl, cycloalkyl aryl, heteroaryl, acyl, amide or carbamate; and
W is N or C-R¹⁰; X is N or C-R¹⁰; Y is N or C-R¹⁰; and Z is N or C-R¹⁰; wherein each R¹⁰ is independently H, halo, alkyl, haloalkyl, alkoxy or heterocyclyl; or
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group.

Embodiment 16 relates to the compound of Embodiment 15, wherein X¹ is -(CH₂)ₙ-, wherein n is 0, 1, or 2.

Embodiment 17 relates to the compound of Embodiments 15-16, wherein R¹⁰ is alkoxy of the formula -OR¹¹, wherein R¹¹ is alkyl, cycloalkyl or aryl, heteroaryl, acyl, amide or carbamate.

Embodiment 18 relates to the compound of Embodiments 15-17, wherein the compound is a compound of the formula:

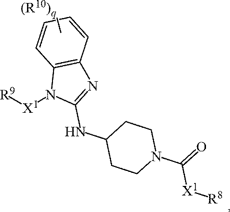

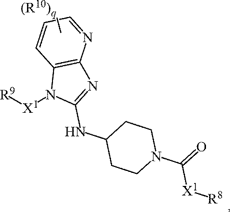

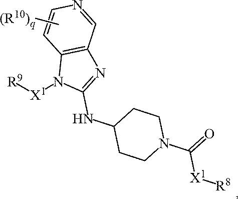

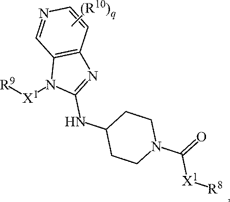

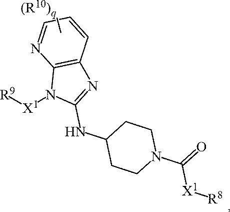

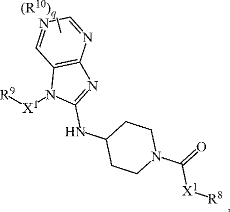

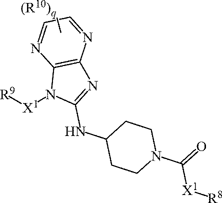

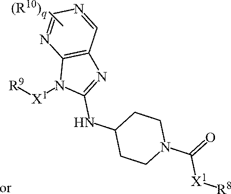

or a pharmaceutically acceptable salt, polymorph, prodrug, solvate or clathrate thereof.

Embodiment 19 relates to the compound of Embodiments 15-18, wherein the compound is a compound wherein R⁹ and R¹⁰, together with the atoms to which they are attached, can form a heterocylyl group:

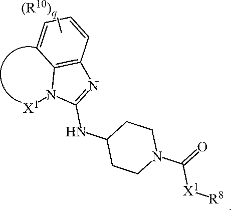

or a pharmaceutically acceptable salt, polymorph, prodrug, solvate or clathrate thereof.

Embodiment 20 relates to the compound of Embodiments 15-18, wherein the compound is a compound of the formula:

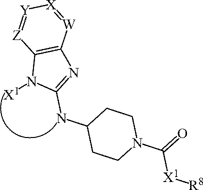

or a pharmaceutically acceptable salt, polymorph, prodrug, solvate or clathrate thereof.

Embodiment 21 relates to the compound of Embodimen 15, wherein the compound is a compound of the formula:

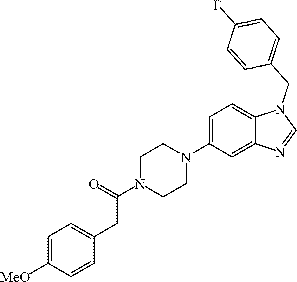 [SMILES: COc1ccc(CC(=O)N2CCN(c3ccc4c(c3)ncn4Cc3ccc(F)cc3)CC2)cc1]

or a pharmaceutically acceptable salt, polymorph, prodrug, solvate or clathrate thereof.

Embodiment 22 relates to a compound of the formula (lll):

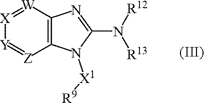

or a pharmaceutically acceptable salt, polymorph, prodrug, solvate or clathrate thereof, wherein:
q is 0, 1, 2 or 3;
X¹ is alkyl or alkenyl;
W is N or C-R¹⁰_{;} X is N or C-R¹⁰; Y is N or C-R¹⁰; and Z is N or C-R¹⁰; wherein each R¹⁰ is independently H, halo, alkyl, haloalkyl, alkoxy or heterocyclyl; or
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group; and
R¹² and R¹³ are each independently alkyl, cycloalkyl aryl, heteroaryl, acyl, amide or carbamate.

Embodiment 23 relates to the compound of Embodiment 22, wherein X¹ is -(CH₂)ₙ-, wherein n is 0, 1, or 2.

Embodiment 24 relates to the compound of Embodiments 22-23, wherein R¹⁰ is alkoxy of the formula -OR¹¹, wherein R¹¹ is alkyl, cycloalkyl or aryl, heteroaryl, acyl, amide or carbamate.

Embodiment 25 relates to the compound of Embodiments 22-24, wherein the compound is a compound of the formula:

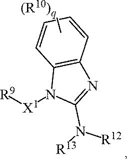

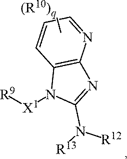

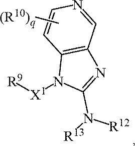

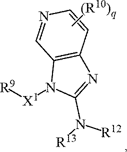

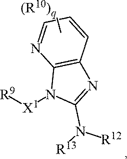

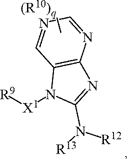

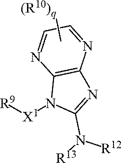

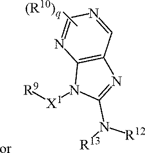

or a pharmaceutically acceptable salt, polymorph, prodrug, solvate or clathrate thereof.

Embodiment 26 relates to the compound of Embodiments 22-25, wherein the compound is a compound wherein R⁹ and R¹⁰ or R⁹ and R¹³, together with the atoms to which they are attached, form a heterocylyl group:

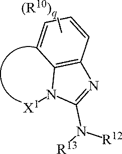

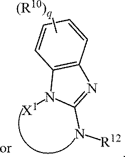

or a pharmaceutically acceptable salt, polymorph, prodrug, solvate or clathrate thereof.

Embodiment 27 relates to the compound of Embodiments 22-26, wherein the compound is a compound of the formula:

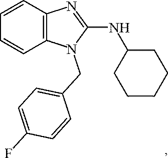

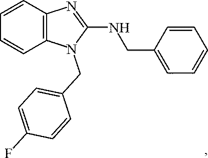

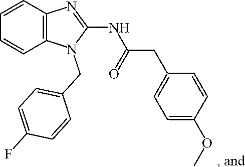

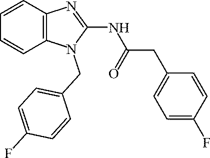 [SMILES: O=C(Cc1ccc(F)cc1)Nc1nc2ccccc2n1Cc1ccc(F)cc1]

or a pharmaceutically acceptable salt, polymorph, prodrug, solvate or clathrate thereof.

Embodiment 28 relates to a compound of the formula (IV):

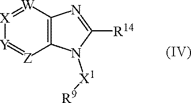

or a pharmaceutically acceptable salt, polymorph, prodrug, solvate or clathrate thereof, wherein:
q is 0, 1, 2 or 3;
X¹ is alkyl or alkenyl;
W is N or C-R¹⁰; X is N or C-R¹⁰; Y is N or C-R¹⁰; and Z is N or C-R¹⁰; wherein each R¹⁰ is independently H. halo, alkyl, haloalkyl, alkoxy or heterocyclyl; or
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group; and
R¹⁴ is aryl or heterocyclyl; or
R⁹ and R¹⁴, together with the atoms to which they are attached, can forma an aryl or a hetorcyclyl.

Embodiment 29 relates to the compound of Embodiment 28, wherein X¹ is —(CH₂)ₙ—, wherein n is 0, 1, or 2.

Embodiment 30 relates to the compound of Embodiments 28-29, wherein R¹⁰ is alkoxy of the formula —OR¹¹, wherein R¹¹ is alkyl, cycloalkyl or aryl, heteroaryl, acyl, amide or carbamate.

Embodiment 31 relates to the compound of Embodiments 28-30, wherein the compound is a compound of the formula:

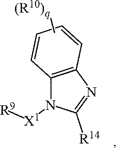

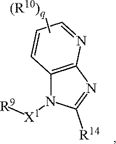

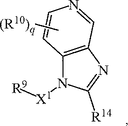

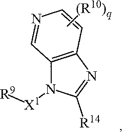

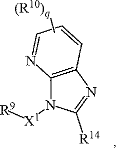

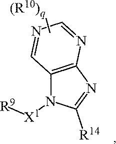

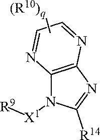

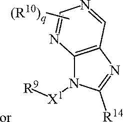

or a pharmaceutically acceptable salt, polymorph, prodrug, solvate or clathrate thereof.

Embodiment 32 relates to the compound of Embodiments 28-31, wherein the compound is a compound wherein R⁹ and R¹⁰ or R⁹ and R¹⁴, together with the atoms to which they are attached, form a heterocylyl group:

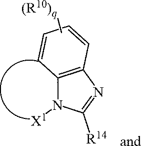

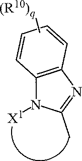

or a pharmaceutically acceptable salt, polymorph, prodrug, solvate or clathrate thereof.

Embodiment 33 relates to a compound of the formula (V):

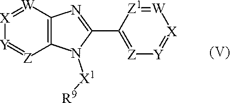

or a pharmaceutically acceptable salt, polymorph, prodrug, solvate or clathrate thereof, wherein:
q is 0, 1, 2 or 3;
X¹ is alkyl or alkenyl;
Z¹ is absent, N or C-R¹⁰;
each W is N or C-R¹⁰; each X is N or C-R¹⁰; each Y is N or C-R¹⁰; and each Z is N or C-R¹⁰; wherein each R¹⁰ is independently H, halo, alkyl, haloalkyl, alkoxy or heterocyclyl; and
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group.

Embodiment 34 relates to the compound of Embodiment 33, wherein X¹ is -(CH₂)ₙ-, wherein n is 0, 1, or 2.

Embodiment 35 relates to the compound of Embodiments 33-34, wherein R¹⁰ is alkoxy of the formula -OR¹¹, wherein R¹¹ is alkyl, cycloalkyl or aryl, heteroaryl, acyl, amide or carbamate.

Embodiment 36 relates to the compound of Embodiments 33-35, wherein the compound is a compound of the formula

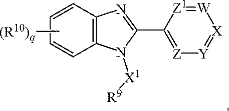

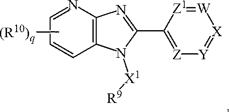

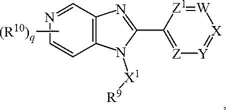

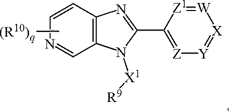

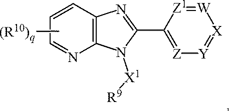

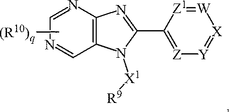

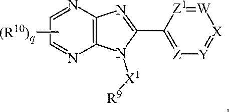

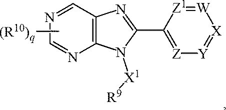

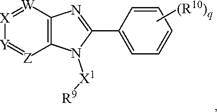

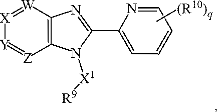

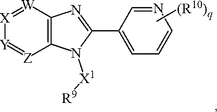

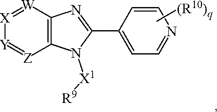

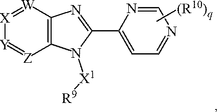

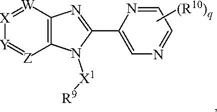

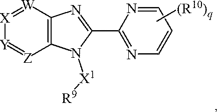

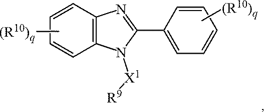

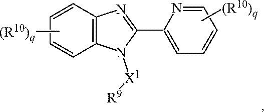

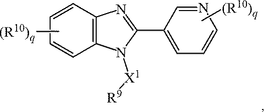

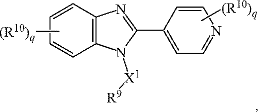

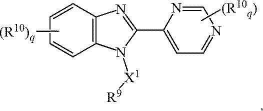

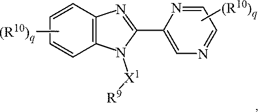

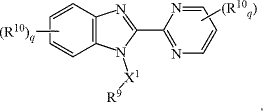

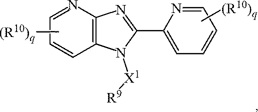

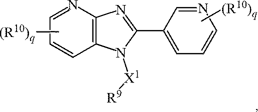

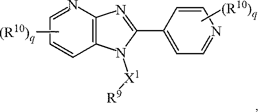

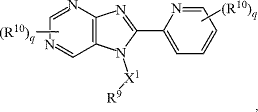

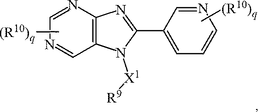

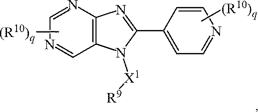

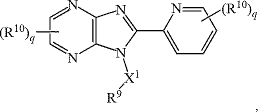

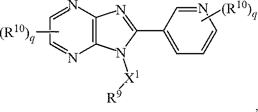

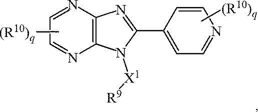

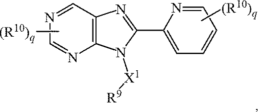

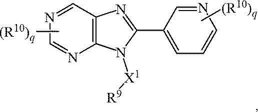

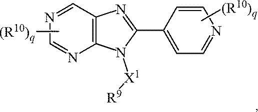

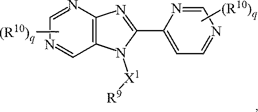

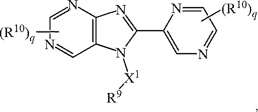

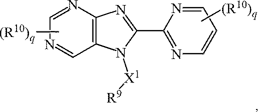

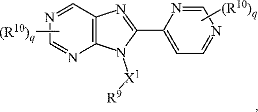

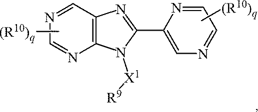

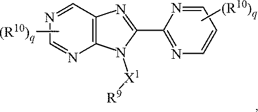

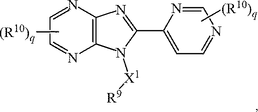

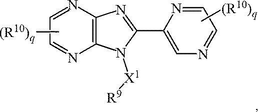

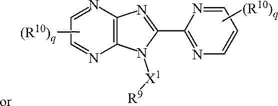

or a pharmaceutically acceptable salt, polymorph, prodrug, solvate or clathrate thereof.

Embodiment 37 relates to the compound of Embodiments 33-36, wherein the compound is a compound wherein R⁹ and R¹⁰ form a heterocylyl group:

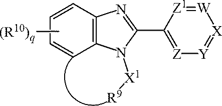

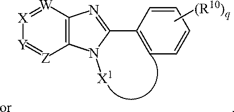

or a pharmaceutically acceptable salt, polymorph, prodrug, solvate or clathrate thereof.

Embodiment 38 relates to the compound of Embodiment 33, wherein the compound is a compound of the formula:

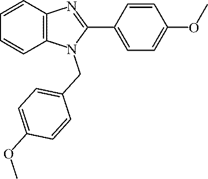 [SMILES: COc1ccc(Cn2c(-c3ccc(OC)cc3)nc3ccccc32)cc1]

or a pharmaceutically acceptable salt, polymorph, prodrug, solvate or clathrate thereof.

Embodiment 39 relates to the compound of Embodiment 33, wherein the compound is a compound of the formula:

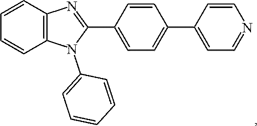

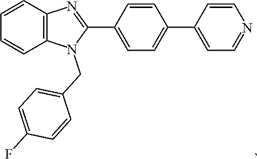

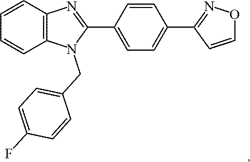

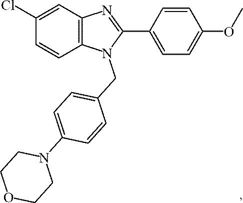

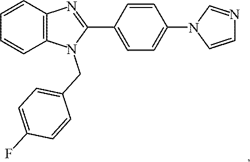

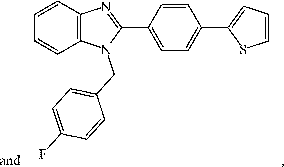

or a pharmaceutically acceptable salt, polymorph, prodrug, solvate or clathrate thereof.

Embodiment 40 relates to a pharmaceutical composition comprising one or more compounds of Embodiments 1-39 and one or more pharmaceutically acceptable excipients.

Embodiment 41 relates to a method for treating a neurodegenerative disease comprising administering a therapeutically effective amount of astemizole, at least one compound of Embodiments 1-39 or a pharmaceutical composition of Embodiment 40 to a subject in need thereof.

Embodiment 42 relates to the method of Embodiment 40, wherein the neurodegenerative disease is at least one of Parkinson’s disease, Alzheimer’s disease, Huntington’s disease, and ALS.

Embodiment 43 relates to a method for reducing, substantially eliminating or eliminating dysregulation of proteostasis comprising administering a therapeutically effective amount of astemizole, at least one compound of Embodiments 1-39 or a pharmaceutical composition of Embodiment 40 to a subject in need thereof.

Embodiment 44 relates to a method for reducing, substantially eliminating or eliminating the accumulation of intrinsically disordered proteins comprising administering a therapeutically effective amount of astemizole, at least one compound of Embodiments 1-39 or a pharmaceutical composition of Embodiment 40 to a subject in need thereof.

Embodiment 45 relates to the method of Embodiment 44, wherein the intrinsically disordered proteins comprise α-syn.

What is claimed is:

1. A compound of the formula (I): 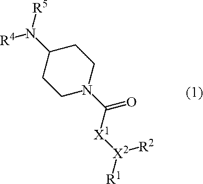
or a pharmaceutically acceptable salt, polymorph, prodrug, solvate or clathrate thereof, wherein:
R¹ is aryl or heteroaryl;
R² is H, alkyl, aryl or heteroaryl;
X¹ is alkyl or alkenyl;
X² is N or CR³, wherein R³ is absent, hydrogen, alkyl, heterocyclyl, or aryl;
R⁴ is hydrogen, alkyl, cycloalkyl, heterocyclyl, aryl or heteroaryl; and
R⁵ is hydrogen, alkyl, cycloalkyl, heterocyclyl, aryl or heteroaryl.

2. (canceled)

3. (canceled)

4. (canceled)

5. (canceled)

6. (canceled)

7. (canceled)

8. (canceled)

9. (canceled)

10. The compound of claim 1, wherein the compound of formula (I) is;
a compound of the formula (Ia): 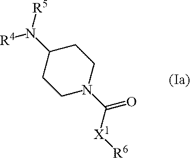
or a pharmaceutically acceptable salt, polymorph, prodrug, solvate or clathrate thereof; wherein:
R⁶ is aryl or heteroaryl; or.
a compound of the formula (Ib) or (Ic): 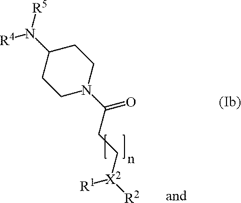 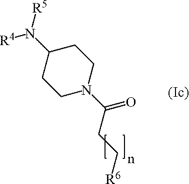
or a pharmaceutically acceptable salt, polymorph, prodrug, solvate or clathrate thereof, wherein:
n is 0, 1 or 2.

11. (canceled)

12. The compound of claim 1, wherein the compound of formula (I) is a compound of the formula: 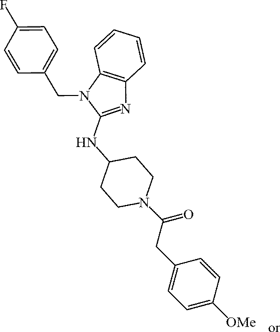 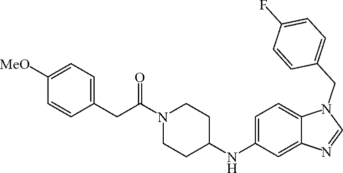 [SMILES: COc1ccc(CC(=O)N2CCC(Nc3ccc4c(c3)ncn4Cc3ccc(F)cc3)CC2)cc1]
pharmaceutically acceptable salts, polymorphs, prodrugs, solvates or clathrates thereof.

13. A compound of the formula: 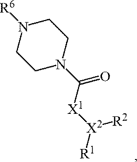 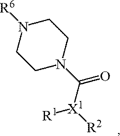 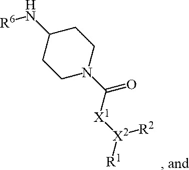 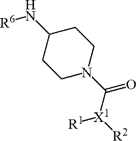
or a pharmaceutically acceptable salt, polymorph, prodrug, solvate or clathrate thereof, wherein:
R¹ is aryl or heteroaryl;
R² is H, alkyl, aryl or heteroaryl;
X¹ is alkyl or alkenyl;
X² is N or CR³, wherein R³ is absent, hydrogen, alkyl, heterocyclyl, or aryl; and
R⁶ is aryl or heteroaryl.

14. (canceled)

15. A compound of the formula (II) and (IIa)-(IId): 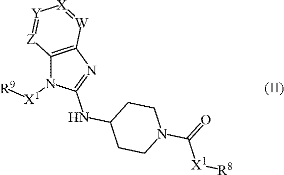 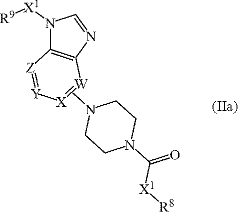 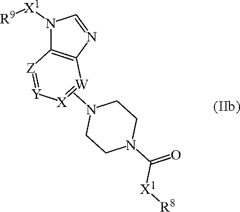 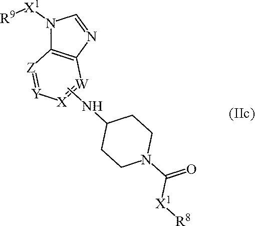 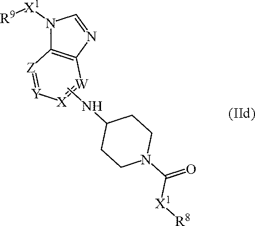
or a pharmaceutically acceptable salt, polymorph, prodrug, solvate or clathrate thereof, wherein:
q is 0, 1, 2 or 3;
each X¹ is independently alkyl or alkenyl;
R⁸ and R⁹ are each independently alkyl, cycloalkyl aryl, heteroaryl, acyl, amide or carbamate; and
W is N or C-R¹⁰; X is N or C-R¹⁰; Y is N or C-R¹⁰; and Z is N or C-R¹⁰; wherein each R¹⁰ is independently H, halo, alkyl, haloalkyl, alkoxy or heterocyclyl; or
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group.

16. (canceled)

17. (canceled)

18. (canceled)

19. (canceled)

20. (canceled)

21. The compound of claim 15, wherein the compound is a compound of the formula: 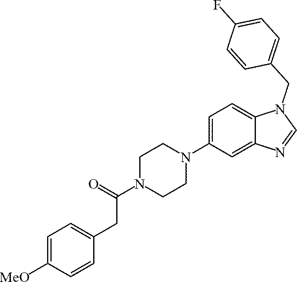 [SMILES: COc1ccc(CC(=O)N2CCN(c3ccc4c(c3)ncn4Cc3ccc(F)cc3)CC2)cc1]
or a pharmaceutically acceptable salt, polymorph, prodrug, solvate or clathrate thereof.

22. A compound of the formula (III): 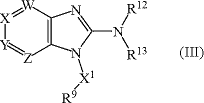
or a pharmaceutically acceptable salt, polymorph, prodrug, solvate or clathrate thereof, wherein:
q is 0, 1, 2 or 3;
X¹ is alkyl or alkenyl;
W is N or C-R¹⁰; X is N or C-R¹⁰; Y is N or C-R¹⁰; and Z is N or C-R¹⁰; wherein each R¹⁰ is independently H, halo, alkyl, haloalkyl, alkoxy or heterocyclyl; or
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group; and
R¹² and R¹³ are each independently alkyl, cycloalkyl aryl, heteroaryl, acyl, amide or carbamate.

23. The compound of claim 22, wherein X¹ is -(CH₂)ₙ-, wherein n is 0, 1, or 2.

24. The compound of claim 22, wherein R¹⁰ is alkoxy of the formula -OR¹¹, wherein R¹¹ is alkyl, cycloalkyl or aryl, heteroaryl, acyl, amide or carbamate.

25. The compound of claim 22, wherein the compound is a compound of the formula: 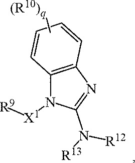 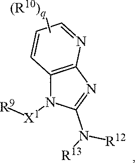 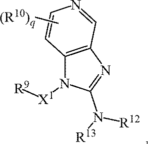 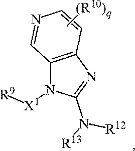 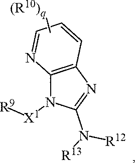 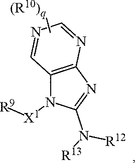 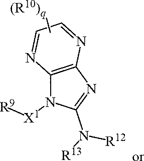 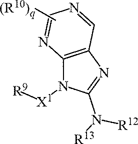
or a pharmaceutically acceptable salt, polymorph, prodrug, solvate or clathrate thereof.

26. The compound of claim 22, wherein the compound is a compound wherein R⁹ and R¹⁰ or R⁹ and R¹³ together with the atoms to which they are attached, form a heterocylyl group: 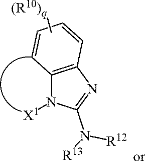 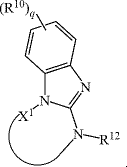
or a pharmaceutically acceptable salt, polymorph, prodrug, solvate or clathrate thereof.

27. The compound of claim 22, wherein the compound is a compound of the formula: 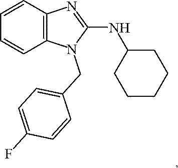 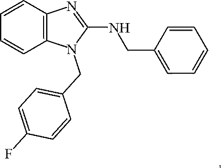 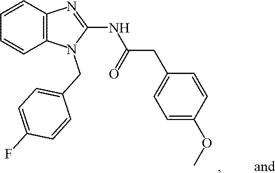 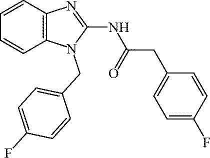 [SMILES: O=C(Cc1ccc(F)cc1)Nc1nc2ccccc2n1Cc1ccc(F)cc1]
or a pharmaceutically acceptable salt, polymorph, prodrug, solvate or clathrate thereof.

28. A compound of the formula (IV): 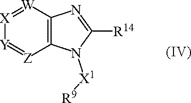
or a pharmaceutically acceptable salt, polymorph, prodrug, solvate or clathrate thereof, wherein:
q is 0, 1, 2 or 3;
X¹ is alkyl or alkenyl;
W is N or C-R¹⁰; X is N or C-R¹⁰; Y is N or C-R¹⁰; and Z is N or C-R¹⁰; wherein each R¹⁰ is independently H, halo, alkyl, haloalkyl, alkoxy or heterocyclyl; or
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group; and
R¹⁴ is aryl or heterocyclyl; or
R⁹ and R¹⁴, together with the atoms to which they are attached, can form an aryl or a hetorcyclyl.

29. (canceled)

30. (canceled)

31. The compound of claim 28, wherein the compound is a compound of the formula: 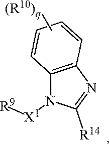 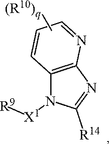 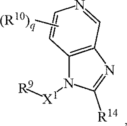 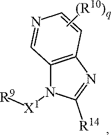 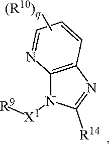 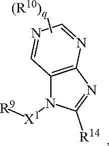 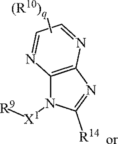 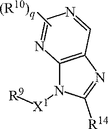
or a pharmaceutically acceptable salt, polymorph, prodrug, solvate or clathrate thereof.

32. The compound of claim 28, wherein the compound is a compound wherein R⁹ and R¹⁰ or R⁹ and R¹⁴, together with the atoms to which they are attached, form a heterocylyl group: 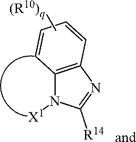 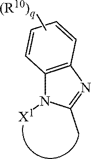
or a pharmaceutically acceptable salt, polymorph, prodrug, solvate or clathrate thereof.

33. A compound of the formula (V): 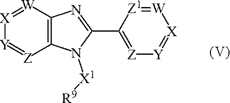
or a pharmaceutically acceptable salt, polymorph, prodrug, solvate or clathrate thereof, wherein:
q is 0, 1, 2 or 3;
X¹ is alkyl or alkenyl;
Z¹ is absent, N or C-R¹⁰;
each W is N or C-R¹⁰; each X is N or C-R¹⁰; each Y is N or C-R¹⁰; and each Z is N or C-R¹⁰;
wherein each R¹⁰ is independently H, halo, alkyl, haloalkyl, alkoxy or heterocyclyl; and
two R¹⁰ groups on adjacent carbon atoms, together with those carbon atoms, can form a cycloalkenyl, aryl or heterocyclyl; or
R⁹ and an R¹⁰, together with the atoms to which they are attached, can form a heterocylyl group.

34. (canceled)

35. (canceled)

36. The compound of claim 33, wherein the compound is a compound of the formula 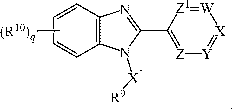 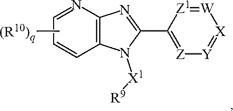 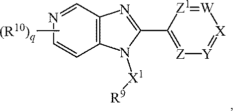 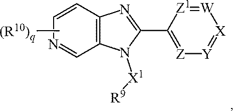 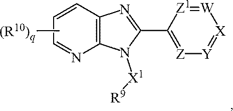 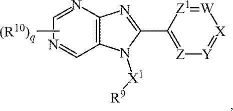 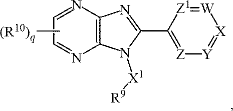 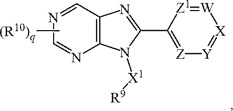 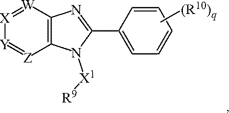 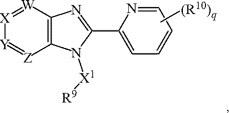 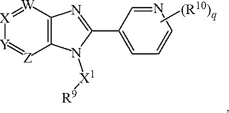 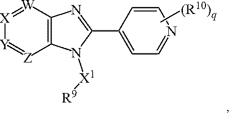 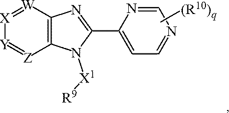 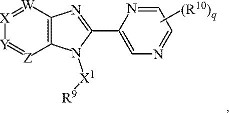 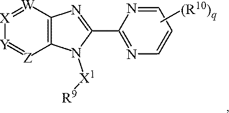 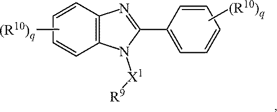 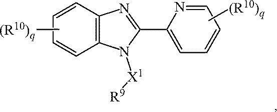 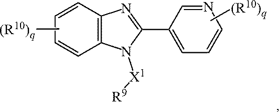 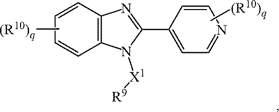 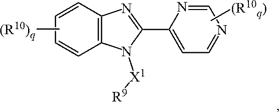 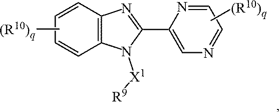 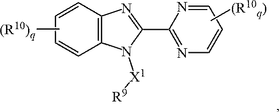 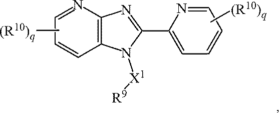 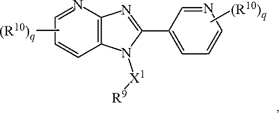 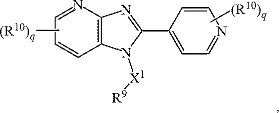 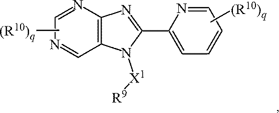 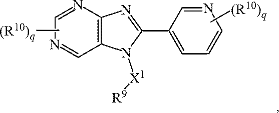 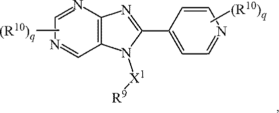 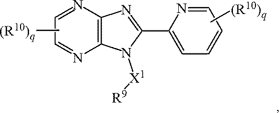 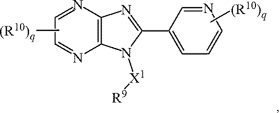 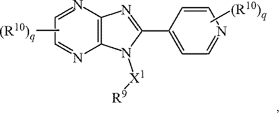 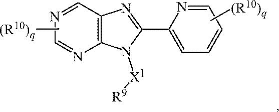 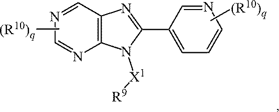 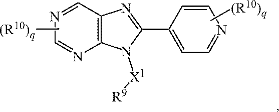 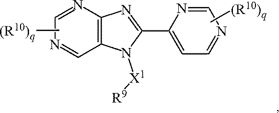 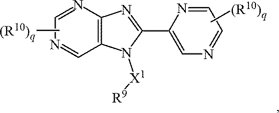 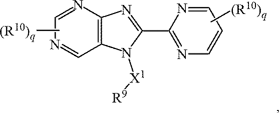 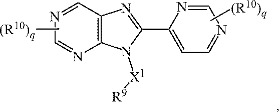 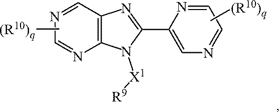 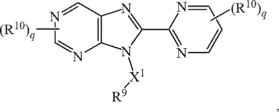 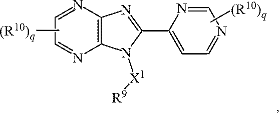 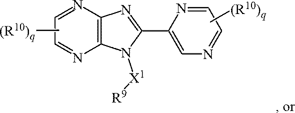 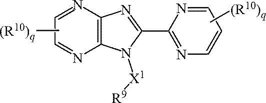
or a pharmaceutically acceptable salt, polymorph, prodrug, solvate or clathrate thereof.

37. (canceled)

38. The compound of claim 33, wherein the compound is a compound of the formula: 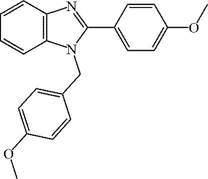 [SMILES: COc1ccc(Cn2c(-c3ccc(OC)cc3)nc3ccccc32)cc1] 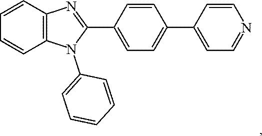 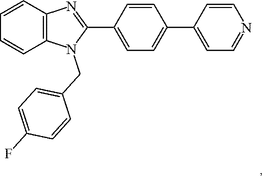 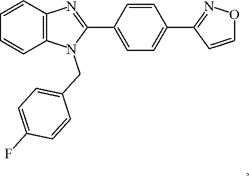 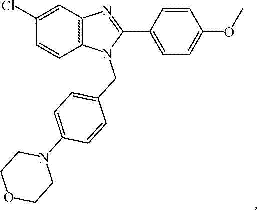 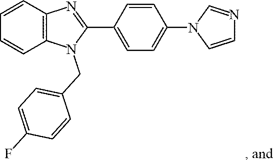 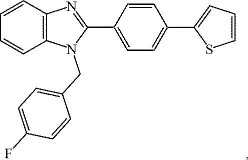
or a pharmaceutically acceptable salt, polymorph, prodrug, solvate or clathrate thereof.

39. (canceled)

40. A pharmaceutical composition comprising at least one of astemizole and one or more compounds of claim 1 and one or more pharmaceutically acceptable excipients.

41. A method for (i) treating a neurodegenerative disease; (ii) reducing, substantially eliminating or eliminating dysregulation of proteostasis; or reducing, or (iii) substantially eliminating or eliminating accumulation of intrinsically disordered proteins comprising administering a therapeutically effective amount of at least one of astemizole and at least one compound of claim 1 to a subject in need thereof.

42. (canceled)

43. (canceled)

44. (canceled)

45. (canceled)

46. (canceled)

47. (canceled)

48. (canceled)

49. (canceled)

50. (canceled)
